(19) United States
(12) Patent Application Publication
(10) Pub. No.: US 2017/0100029 A1
(43) Pub. Date: Apr. 13, 2017

(54) Compositions and Methods for Analyzing Collateral Density

(71) Applicant: The University of North Carolina at Chapel Hill, Chapel Hill, NC (US)

(72) Inventor: James E. Faber, Chapel Hill, NC (US)

(21) Appl. No.: 15/292,998

(22) Filed: Oct. 13, 2016

Related U.S. Application Data
(60) Provisional application No. 62/240,752, filed on Oct. 13, 2015.

(51) Int. Cl. A61B 3/00; G06K 9/00; A61B 3/14; A61B 5/00; A61B 5/02; A61B 3/12

(52) U.S. Cl. CPC A61B 3/0025; A61B 5/02007; A61B 3/1241; A61B 3/14; A61B 5/7275; G06K 9/0061; G06K 9/00617

(57) ABSTRACT

The present invention provides a retinal predictor index (RPI), composed of discrete geometric and fractal descriptors of the branch-patterning of the outer retinal circulation, as a biomarker for differences in the extent (number and diameter) of collateral blood vessels in brain, heart, lower extremities and other tissues.

### STATEMENT OF PRIORITY

This application claims the benefit, under 35 U.S.C. §119(e), of U.S. Provisional Application No. 62/240,752, filed Oct. 13, 2015, the entire contents of which are incorporated by reference herein.

### STATEMENT OF GOVERNMENT SUPPORT

This invention was made with government support under Grant Nos. HL090655, HL111070, NS083633 and T35-DK007386, awarded by the National Institutes of Health. The government has certain rights in the invention.

### FIELD OF THE INVENTION

The present invention relates to a retinal predictor index (RPI), composed of discrete geometric and fractal descriptors of the branch-patterning of the outer retinal circulation, as a biomarker for differences in the extent (number and diameter) of collateral blood vessels in brain, heart, lower extremities and other tissues.

### BACKGROUND OF THE INVENTION

Occlusive vascular disease in brain, heart and peripheral limbs is caused by atherosclerosis, thrombosis and other disorders and imposes large social and economic burdens. Although available treatment options (e.g., thrombolysis, thrombectomy, angioplasty/stent, bypass grafting) are successful in many patients, time window for treatment, co-morbidities, inaccessibility and diffuse obstructions exclude large numbers of patients. Irrespective of treatment availability or type, an abundant collateral circulation greatly reduces morbidity and mortality in these diseases. Collaterals are native (pre-existing) arteriole-to-arteriole anastomoses that cross-connect a small fraction of the outer branches of adjacent arterial trees and are present in most tissues. When the trunk of one of the trees becomes obstructed, collateral-dependent retrograde perfusion significantly decreases tissue injury. The amount of protection depends primarily on the extent (i.e., number and diameter) of collaterals present, plus the perfusion pressure across the collateral network and vascular resistance above and below it.

Collateral extent in tissues varies widely among individuals from naturally occurring differences in genetic background as do differences in the retinal circulation's vascular trees. Subjects with low (poor) collateral extent are predisposed toward increased severity of tissue injury and loss resulting from thrombi, emboli, atherosclerosis and other types of arterial obstruction diseases and conditions.

The major determinants of severity of ischemic stroke are site of occlusion within the tree, time to endovascular revascularization when this is an option, and collateral blood flow. Unfortunately, collateral perfusion varies widely among individuals in brain, heart, lower extremities and other tissues. For example, in patients with occlusion of the middle cerebral artery (MCA), the most common cause of ischemic stroke, collateral-dependent perfusion of the MCA tree—which can be graded (scored) by neuroimaging—varies widely, with approximately twenty percent having poor pial (leptomeningeal) collateral scores (i.e., poor collateral “status”). Notably, such individuals sustain larger infarct volumes, respond poorly to thrombolytic treatments, have increased risk for and severity of intracerebral hemorrhage, and suffer increased morbidity and mortality. Recent studies have confirmed a similar wide variation in collateral blood flow in other tissues among “healthy” humans, i.e., without obstructive disease in the tissue under examination. In individuals without angiographically detectable coronary artery disease (CAD), collateral flow index (CFI) was distributed normally and varied by ˜25-fold, with approximately twenty percent of individuals having low CFIs. Importantly, patients with CAD and poor CFIs had a sixty-four percent higher risk of mortality. CFI also varied significantly in the lower extremities of individuals without peripheral artery disease (PAD). Thus, assessing collateral status is increasingly regarded as an important means to identify optimal treatment and assess prognosis for recovery.

A critical problem is that no non-invasive method exists for determining the extent of the collateral circulation in healthy humans or patients with an obstructive disease or condition. Since the diameters of most native collaterals are below the resolution of clinical imaging modalities, measurement of pial collateral score in acute stroke is used to indirectly estimate conductance of the collateral network. This method requires administration of a contrast agent, thus is invasive. It also relies on advanced neuroimaging not available at most treatment centers. Estimation of collateral-dependent perfusion in heart and lower extremities requires temporary intra-arterial balloon occlusion, which generally restricts its use to experimental studies. Thus, a non-invasive method or biomarker that predicts collateral extent would be an important development.

The present invention provides methods and compositions for determining a retinal predictor index (RPI) for a subject, which can be used in guiding treatment of arterial obstruction diseases and/or pathological conditions of the arteries.

### BRIEF DESCRIPTION OF THE DRAWINGS

FIG. 1. Collateral number and diameter vary widely with genetic background and are a major determinant of the accompanying wide variation in infarct volume (ANOVA, p<0.001, n=number of 3-4 month-old male mice of each strain—given below columns). Representative images of neocortex of 2 strains of mice with high and low collateral number and diameter and infarct volume 24 hours after permanent middle cerebral artery ligation.

FIG. 2. Measurement and definitions of retinal patterning metrics (RPM) numbers 1 to 10: retinal area, vessel diameter (D0, D1, D2), optimality, branch angle, tortuosity index (inner zone), central retinal artery equivalent (CRAE), central retinal vein equivalent (CRVE), and artery-to-vein ratio (AVR). Image of flat-mounted, stained (Alexafluor 568 GS-IB4) retina is imported into Photoshop CS4 (panel A). A circle centered on the optic disc and positioned over the outer zone-margin of the retina (OZ) is drawn so its circumference is tangential to the marginal veins (MV) along a majority of the flaps. The optic disc margin (OD) and inner-zone margins (IZ) are drawn at 1/10^{th} and 5/10^{th} of the OZ diameter. The borders of the retina are traced to measure the retinal area (RPM 10). All remaining manually obtained RPMs (numbers 1-4, 8, 9) are measured in the region lying between the OD and IZ from 4 randomly chosen arterial trees (dashed boxes). To calculate inner-zone tortuosity index (RPM 4), a line (l) is traced on each tree along the vessel axis from the OD to IZ margin and a vector line (L) is drawn connecting the endpoints (B). A circle with a 25-pixel radius is manually placed encircling each branch point along the tree between OD and IZ. The center of Bc is approximately placed at the point of intersection (P) of the vessel axes of the Parent and Larger branch. Vessel diameters (Parent-D0, Smaller branch-D1, Larger branch-D2) are measured perpendicular to the axis and used to calculate the optimality ratio; (RPM 9) branch angle is the angle of the rays bisecting the diameters (C). The above methods ensure consistent measurement of RPMs. All numbers obtained from the 4 trees are averaged to obtain a mean of each RPM for each mouse. CRAE and CRVE are measured using the revised Parr-Hubbard formulas and combined to obtain AVR. Using ImageJ, vessel calibers of retinal arterioles and venules were measured between 0.5 and 1 optic disc diameters from the OD and summarized into the CRAE and CRVE (D). Six of the largest arterioles and six of the largest venules are identified, and using an iterative procedure, the largest and smallest calibers are paired and entered into their respective formulas to obtain 3 vessel calibers (E). This procedure is repeated to obtain 2 and finally 1 central vessel equivalent, i.e., the CRAE and CRVE and AVR (RPMs 5-7). Definitions of retinal patterning metrics (RPM) numbers 1 to 10: 1, 2, & 3. D0, D1, and D2 (μm)—Diameter of the parent and branching daughter vessels; 4. Tortuosity index (inner zone)—Ratio of scalar and vector length of the segment (1/L); 8. Branch angle—Angle between daughter vessels that bisects Dₒ; 9. Optimality—Measure of equitability of distribution of flow from parent to daughter vessels:

$$\sqrt[3]{\frac{{D}_{1}^{3} + {D}_{2}^{3}}{2 {D}_{0}^{3}}} ;$$

10. Retinal area (μm²)—Area of retina encompassed by white dashed tracing in A; 5. Central retinal artery equivalent (CRAE)—Estimated caliber of the central retinal artery; 6. Central retinal vein equivalent (CRVE)—Estimated caliber of the central retinal vein; 7. Artery-to-vein ratio (AVR)—The ratio of CRAE and CRVE.

FIG. 3. Measurement and definitions of semi-automatically obtained retinal patterning metrics (RPMs) numbers 11 to 22. Each artery tree was binarized using ImageJ (A) and skeletonized using the Analyze Skeleton plugin (B). Each tree is composed of branch segments defined as the length of tree lying between two branch points (B). Functions within ImageJ are used to obtain RPMs 13 and 14 from the binarized image, while the Skeletonize plugin is used to calculate scalar length (l), vector length (L), and tortuosity of each branch segment (B). Distributions (average, skew, and kurtosis) of these values are calculated using Microsoft Excel to obtain RPMs 17-22 for each arterial tree (C, D). A MATLAB program is used to calculate fractal dimension and lacunarity using the sliding box-count method (E). All numbers obtained from the 2-3 randomly chosen trees (FIG. 9) are averaged to obtain a mean of each RPM for each mouse. Definition of retinal patterning metrics (RPM) Numbers 11 to 22: 11, 12. Fractal dimension and Lacunarity—Dimensionless measures of vessel complexity; boxes of specific sizes slide across the image and count pixels that are encountered. The slope of the log of the mean (or coefficient of variation) of pixel count versus box size is used to calculate fractal dimension (or lacunarity) (E); 13. Arterial tree area (μm²)—Total area of binarized tree (A); 14. Skeletonized arterial tree area (μm)—Total area of skeletonized tree, i.e., total length of arterial tree segments; 15. Average arterial tree diameter (μm)—Ratio of metric obtained from 13 and 14; 16. Number of arterial tree branch segments/tree area (μm²)—Ratio of total number of branch segments in the tree and metric obtained in 13; 17. Average tortuosity of branch segments—Average tortuosity (B) of all branch segments in the tree; 18. Skewness of distribution of branch segment tortuosity—Asymmetry of distribution of branch segment tortuosity; more positive (or negative) skew results from higher proportion of segments with high (or low) tortuosity (C); 19. Kurtosis of distribution of branch segment tortuosity—Peakedness (or flatness) of distribution of branch segment tortuosity; more positive (or negative) kurtosis means a distribution that is steeper (or flatter) compared to a normal distribution. Higher kurtosis results from a greater proportion of segments with similar tortuosity centered around average (C); 20. Average length of branch segments (μm)—The average scalar length, 1 of all branch segments (B); 21. Skewness of distribution of branch segment lengths—Asymmetry of distribution of branch segment lengths; more positive (or negative) skew results from higher proportion of segments with high (or low) length (D); 22. Kurtosis of distribution of branch segment lengths—Peakedness (or flatness) of distribution of branch segment length; higher kurtosis results from a greater proportion of segments with similar length centered closer to the average (D).

FIG. 4. Summary of study design and statistical and outlier analysis. Collateral number and diameter (COL-N and COL-D) and 22 RPMs were obtained from 81 mice ((panel A) and FIGS. 1-3, Table 1, FIG. 9). A preliminary multivariate outlier analysis of all observations found one mouse with significantly high Mahalanobis, jackknife, and T² distances (n=1) (Panels A, B). Bivariate regression (A) of COL-N, COL-D, and RPMs was performed on the remaining 80 mice to determine degree and significance of covariance and correlation with genetic strain (FIG. 5). Forward, backward, and mixed-direction stepwise multivariate regression modeling of RPMs versus COL-N and COL-D was performed using three different criteria—minimum AIC and BIC, and p-value cutoffs—to obtain 7 different models (RM1) (n=80) (FIG. 15). A K-fold R² value for each model was obtained using leave-one-out cross-validation to assess the average predictive performance of the modeling process for each of the 7 models repeated n-times (correlation between actual and predicted COL-N or COL-D for a given model developed on n−1 observations) (FIG. 15). Observations with significantly high Cook's D influence averaged across RM1 of COL-N and -D, COL-N, or COL-D models were identified (C) and excluded to repeat the aforementioned modeling process (RM2 and RM3) ((A) and FIG. 15). The most predictive models for COL-N and COL-D (i.e., combination of RPMs) among RM1, RM2, and RM3 were used to obtain a retinal predictive index—RPIₙ and RPI_{d}, respectively (FIG. 6). Performance of the average RPIₙ and RPI_{d} for each strain in predicting historic average infarct volume for each strain was tested (FIG. 8). In the outlier box plots (B and C), the vertical line within the box represents the mean; top and bottom points of diamond represent the 95% confidence interval of the mean; the ends of the box represent the 25th and 75th quantiles and 3^{rd} quartile (Q)) and span the interquartile range (IQR) of the data; the box-lines that extend from the top and bottom end of the box (whiskers) represent the 1^{st} Q−1.5·(IQR) and 3^{rd} Q+1.5·(IQR), respectively. The bracket outside identifies the most dense 50% of the observations.

FIG. 5. Retinal patterning metrics (RPMs) vary with genetic background and correlate with collateral number (COL-N), diameter (COL-D) and other RPMs. Bivariate regression (one-way ANOVA adjusted R² and p-value) of COL-N, COL-D and RPMs versus mouse strain (panel A) show that COL-N, COL-D and 10 out of 22 RPMs strongly vary with genetic background (black columns, relative strength of correlation; dashed line, adjusted R² of >0.35, p<0.0001). These data suggest that, similar to its contribution to the variation in COL-N and COL-D, genetic background also plays a significant role in specifying variation in features of retinal vascular patterning, such as vessel caliber, branch angle, etc. Multivariate correlation matrices (B and C) of COL-N, COL-D and RPMs show significant covariance; matrices show direction and strength (+/− adjusted R² (B)), and significance (p-value, (C)) of covariance. Highlighted regions of matrices (B,C; upper boxes) show that a number of RPMs (1-3,5,6,10,11,13-15,17,20) also vary strongly with COL-N and COL-D, suggesting that they may also predict collateral extent. Fractal dimension and lacunarity (RPMs 11 and 12), global descriptors of pattern complexity, both correlate with other RPMs and are inversely related to each other (B,C; lower boxes).

FIG. 6. Among the most predictive RPMs, average diameter of larger branch vessel at an arterial tree bifurcation along the length of the retinal arteries emanating from the optic disc (D2) contributes the most, statistically, to predicting collateral number and diameter (COL-N and COL-D). To compare the relative predictive power of the RPMs, parameter estimates from the 2 most predictive models for RPIₙ and RPI_{d} (FIG. 15) were obtained and plotted as scaled estimates (i.e., centered by mean and normalized to have identical range) (A,C); in addition, the parameter estimates were standardized to have equal variances, orthogonalized to be uncorrelated, and plotted—in descending order of scaled estimates—as a pareto plot (B,D). The scaled estimates show the relative extent of change in COL-N or COL-D as a specific RPM is varied from the lowest to highest value in the population of mice. The pareto plot accounts for covariance and extent of variability of an RPM in the mouse population to estimate and arrange the RPMs in descending order of relative “explanatory power.” Plots of predicted COL-N and COL-D versus RPIₙ and RPI_{d}, along with K-fold R² reveal the spread of data and the strength of correlation (E,F). ****P<0.0001.

FIG. 7. Comparison of retinal and pial images of a VEGF^{hi/+} and BALB/c mouse with wide differences in number of MCA-to-ACA collaterals (COL-N)—27 vs. 0 collaterals, respectively. “Branch points,” magnified images of selected branch points (5 small boxes in retina). “Trees,” regions of retinal arterial trees (3 large boxes in retina). Values of predictive retinal metrics for the two mice are shown for comparison. Consistent with the retinal prediction modeling (FIG. 6), the mouse with a high COL-N(27) has a higher RPIₙ (25.6), larger branch vessel caliber (D2), branch angles at branch points, retinal area, and CRAE, but lower lacunarity, shorter branch segments, and lower optimality (i.e., more equitable distribution of vessel calibers of daughter vessels at bifurcations). However, contrary to expectations, this mouse had a larger kurtosis of distribution of branch segment lengths (i.e., higher proportion of branch segments with similar lengths) and larger parent vessel caliber (D0) attributable to both biological variance and experimental error.

FIG. 8. Average retinal predictor index for collateral number and diameter (RPIₙ and RPI_{d}) predict average infarct volume following middle cerebral artery occlusion (MCAO) in 10 mouse strains. Average strain RPIₙ and RPI_{d} predict pial collateral number (COL-N) and (COL-D), respectively (A), which in turn predict average MCAO infarct volume for strains (B, FIG. 1). Accordingly, RPIₙ and RPI_{d} predict average infarct volume after MCAO (C). This indicates that retinal patterning, which predicts COL-N and COL-D, is a strong non-invasive biomarker of infarct volume in ischemic stroke in mouse. K-fold R² values obtained by leave-one-out cross-validation and p-value (p-value***,****<0.001, 0.0001) show strength and significance of correlation between actual and predicted parameters. ***,****P<0.001, 0.0001.

FIG. 9. Retinal tree segmentation. Image of flat-mounted stained retina (A) is segmented using Photoshop CS4, 3 retinal trees are randomly selected and capillaries are manually pruned away using specified segmentation rules so that only 1^{st}, 2^{nd}, 3^{rd}, and the half-length of 4^{th} order arterioles are retained (B). Using a combination of the Leveling tool and optimization of brightness and contrast, background is eliminated, dark or missing segments of trees are filled in and the image is thresholded (C).

FIG. 10. Collateral number, collateral diameter and retinal patterning metrics vary with genetic background. Graphs show averages, 95% confidence intervals, and bivariate regression of COL-N, COL-D, and 22 RPMs versus 10 mouse strains (one-way ANOVA—adjusted R² and p-value are given on the graphs). Horizontal line across each graph represents mean of the metric across all strains. Top and bottom points of diamond represent the 95% confidence interval of the mean for each strain. Diamond width is proportional to the n-size of the strain. Relative variation in vertical position of diamonds reveals degree to which COL-N, COL-D, and RPMs vary across all strains. FIG. 3D shows 1 of 3-4 selected arterial trees chosen randomly from a retina. The distribution displayed for this arterial tree happens to lack branch segments measuring 175-200 microns length, which makes the distribution appear to have two peaks, i.e., one large population of small branches and another smaller population with really large branches. This is an incidental finding secondary to biological variation and experimental error. The consistently positive skewness of distribution of branch segment lengths for all arterial trees reflects continued branching of the central retinal artery into a few large parent trunks which, in turn, further divide into smaller daughters as terminal branching approaches capillaries.

FIG. 11. Among 4 strains of mice with large differences in collateral extent, the range of values is greater and variance (SEMs) smaller for fractal dimension (panel C) determined for a randomly selected region of interest on arterial tree (ROI) (inset in A) than for whole retina (B). Thus, the complexity of the branching pattern of arterial trees likely contains more genetic background-specific features than the capillary bed and venous trees. Removal of the capillaries and venules/veins will therefore increase the statistical power to test for association of retinal vascular patterning metrics with pial collateral number and diameter among genetically distinct strains. A, representative image stained with IB4 lectin and converted to gray-scale. Distal arterial trees were randomly selected and analyzed for an ROI of constant dimension. See FIG. 3 for determination of fractal dimension and lacunarity. Values here and in other on-line figures are means±SEM unless indicated otherwise. Fractal dimension and lacunarity varied with strain (ANOVA, p<0.05). BC, BALB/c strain; BLKS, C57BLKS strain. N=5 mice per strain.

FIG. 12. Fractal dimension of the distal-most region of the vasculature between adjacent artery and vein trees (i.e., the “capillary bed”) lacks strain-dependent differences shown in FIG. 10 for whole retina or individual artery trees. The lower values than in FIG. 11 indicate that patterning of the capillary bed has less complexity than individual arterial trees or whole retina. Fractal dimension was determined for randomly selected ROIs of constant dimension that encompassed the capillary bed between an artery and vein tree. These findings indicate that removal of capillaries from each artery tree during image processing (“pruning”) is required to obtain arterial patterning metrics to test for association with genetic background-dependent differences in collateral number and diameter. A representative ROI showing Alexa-568 isolectin B4 staining is shown above left. N=5 mice per strain. The absence of strain dependent differences in fractal dimension for the capillary bed is consistent with evidence that angiogenesis (capillary formation) is dominated by stochastic processes. The findings in FIGS. 10-12 that the more mesh-like capillary network must be removed to accurately measure fractal dimension of a dichotomously branching artery tree are intuitive. Data at bottom, taken from FIG. 10, shows FD and Lac for fully processed arterial trees used in the main analysis in this study (FIG. 5)—for comparison to the above data. Strains: B—AKR, C—BALB/c, D—C57BLKS, G—DBA/2 (boxes).

FIG. 13. Complexity of genetic-dependent vascular patterning, as indicated by fractal dimension of randomly selected artery trees, is not altered by removal of the distal-most region of the vasculature between adjacent artery and vein; however, variance (SEMs) is reduced. A,B, representative binarized images and summary data after pruning away the venous side of the “capillary” bed back to the capillary midpoints. C,D, images and data of artery trees after pruning away capillaries and distal-most arterioles to just before joining the parent arteriole. Thus, pruning away of capillaries and distal-most arterioles of each retinal artery tree during image processing yields genetic-dependent arterial patterning metrics with the least variance. N=5 mice per strain. *, ** p<0.05, 0.01. BC, BALB/c strain; B6, C57Bl/6 strain. Fractal dimension, lacunarity and the Image J “plug-in” metrics reported in figures and tables in the paper and in the on-line section were obtained from artery tree images processed as shown in FIGS. 2, 9 and 13A,C.

FIG. 14. Comparison of fractal dimension (FD) and lacunarity (Lac) obtained from 3, 2 and 1 randomly chosen arterial tree(s).). Tree segmentation for obtaining semi-automated retinal patterning metrics (FIG. 9) is a labor-intensive process. Average FD and Lac, two dimensionless measures of image complexity (FIG. 3), were measured from 3 randomly chosen tree(s) from 5 mouse retinas each for 4 strains (AKR, BALB/c, C57BLKS, and DBA/2) that vary widely in collateral number (n=20) (Table 1). The above data show a strong correlation between average FD and lacunarity obtained from 3 versus 2 trees (A,C) (adjusted R² of 0.86 and 0.83, respectively), which drops significantly when comparing 3 versus 1 tree (B, D) (adjusted R² of 0.64 and 0.55, respectively). Thus, for the remaining 60 mice in the study, only 2 randomly chosen arterial trees were segmented in order to achieve an optimal trade-off between accuracy and time required for image segmentation.

FIG. 15. Retinal patterning predicts pial collateral number (COL-N) and diameter (COL-D). This figure shows results of forward, backward, and mixed direction stepwise multivariate regression of COL-N and COL-D versus retinal patterning metrics (RPMs) using different stopping rules—minimum AIC, minimum BIC, and p-value cutoffs (p<0.25 for RPM to enter and >0.10 to leave model) before (RM1) and after (RM2 and 3) removal of influential outliers (FIG. 4). Predictive performance across all models (RM1, RM2, and RM3) as determined by K-fold R² calculated from leave-one-out cross validation was strong (0.73-0.83 for COL-N and 0.59-0.73 for COL-D) and confirmed our hypothesis that RPMs can be used to predict COL-N and COL-D. Removal of outliers improved K-fold R² but did not change our conclusion. Directionality, strength, and significance of correlation of RPMs with COL-N and COL-D across all models are displayed and highlight the RPMs that remained predictive throughout all models. 11 of the 16 RPMs found to predict COL-N also predicted and correlated with COL-D in a similar direction, consistent with the covariance of COL-N and COL-D in the mouse population (FIG. 1) suggesting that genetic determinants of variation in collateral extent also influence variation in key features of retinal vascular patterning. The most predictive models based on highest K-fold R² was used to calculate a Retinal Predictor Index for COL-N and COL-D (RPIₙ and RPI_{d}, respectively) and further examined to determine comparative predictive ability of individual RPMs (FIG. 6).

FIG. 16. (A) Differences in fractal dimension (FD) and lacunarity (L) of retinal artery trees are associated with differences in retinal patterning metrics (RPMs). (B-I) Among RPMs characterizing retinal arterial tree complexity, differences in FD and L are most strongly associated with differences in CRAE and average length of branch segments, respectively.

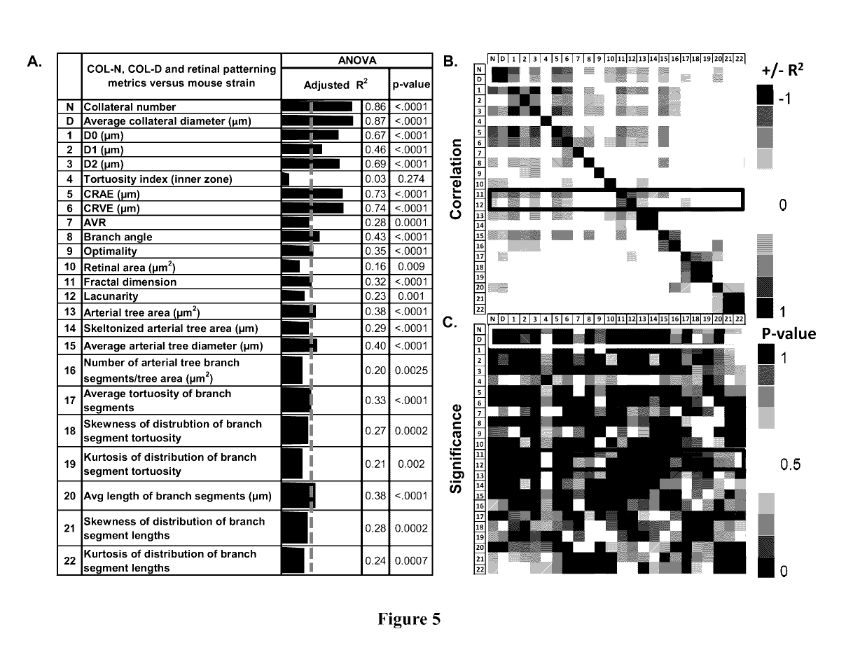

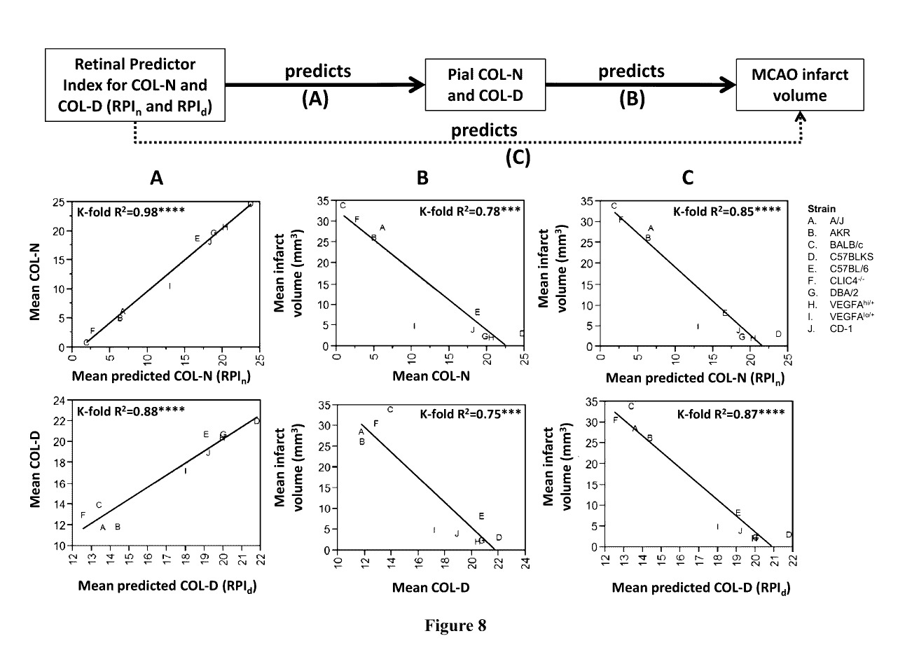

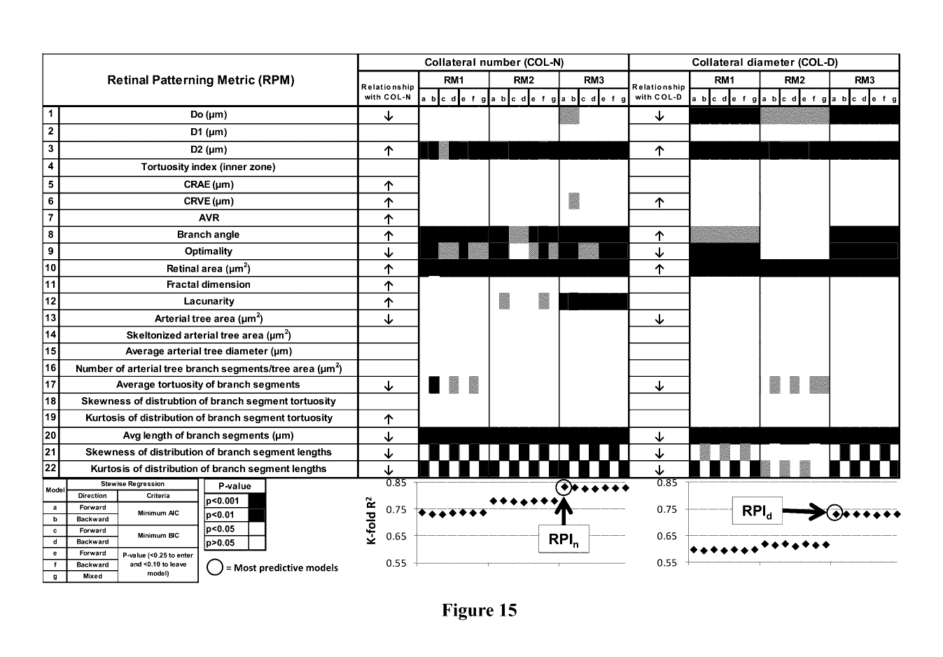

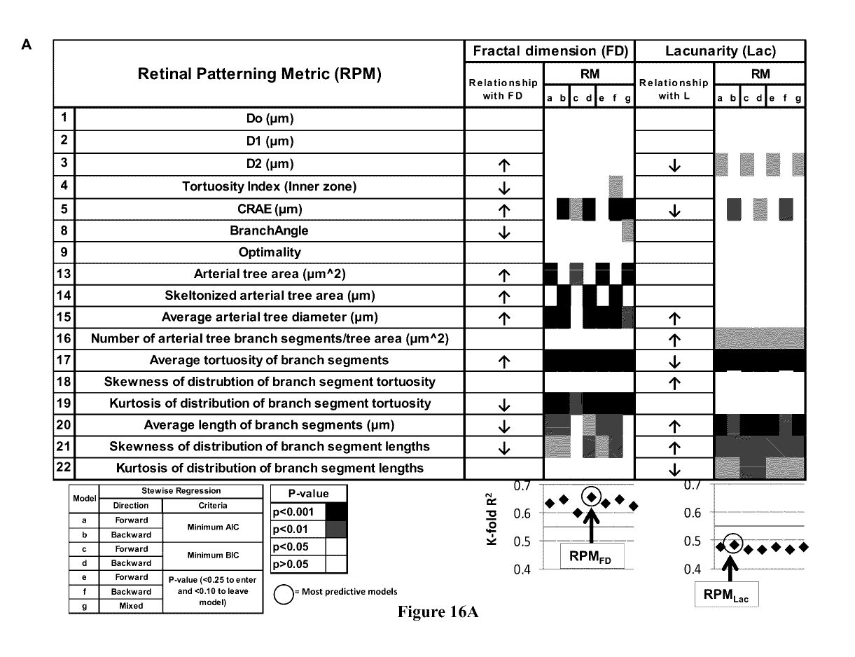

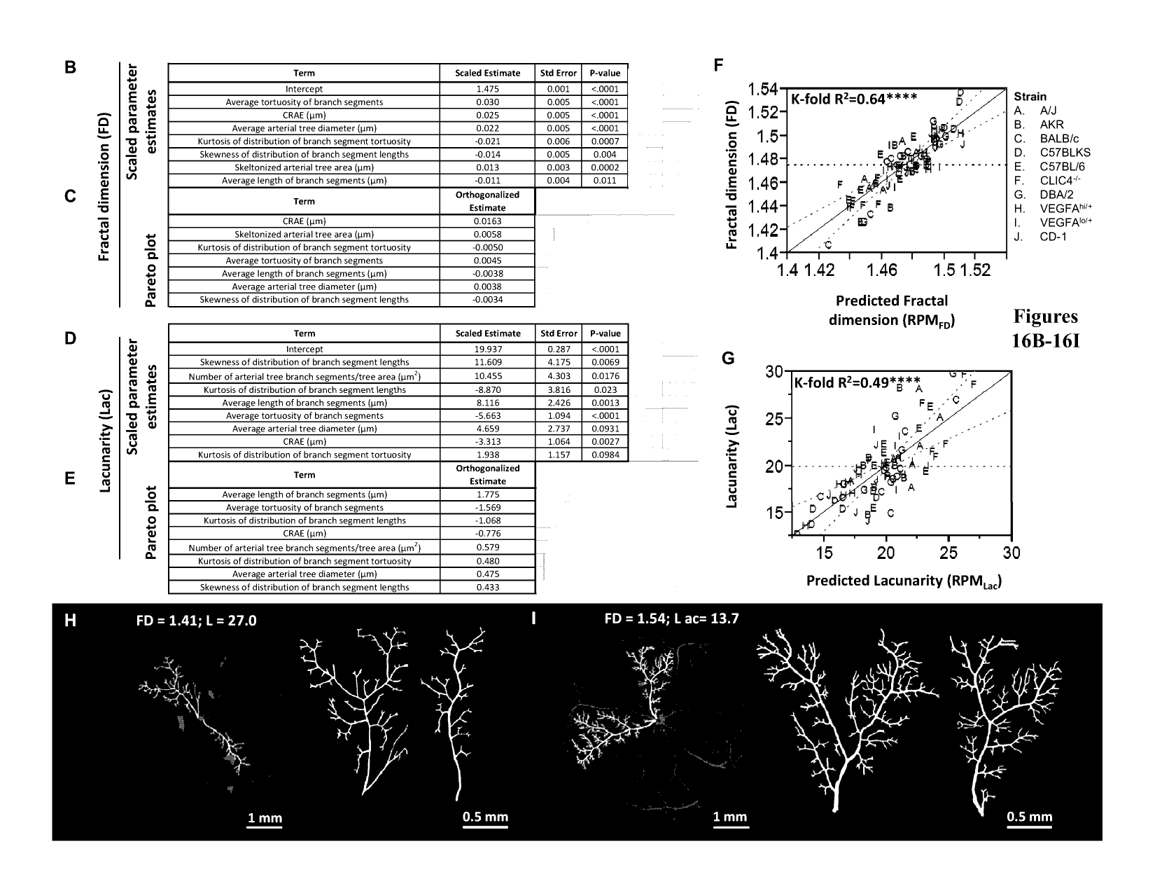

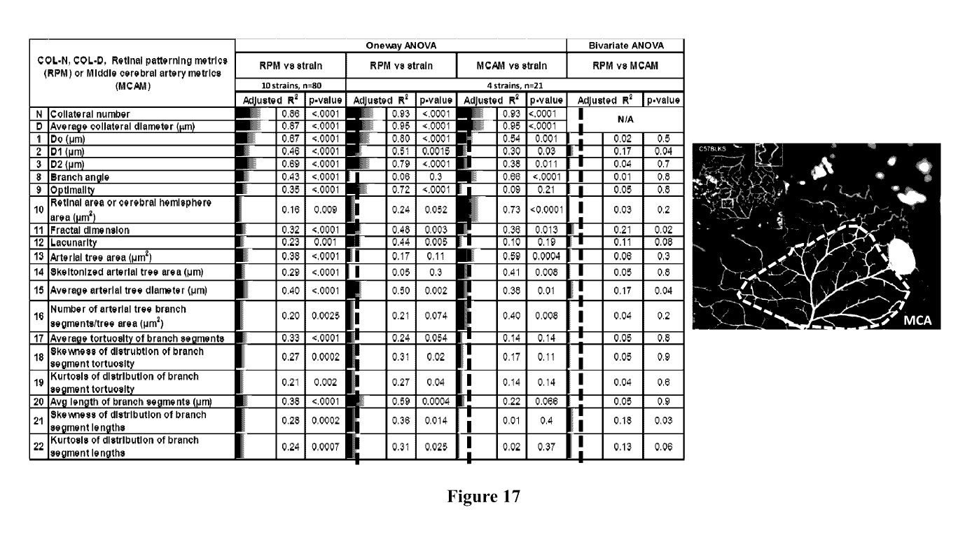

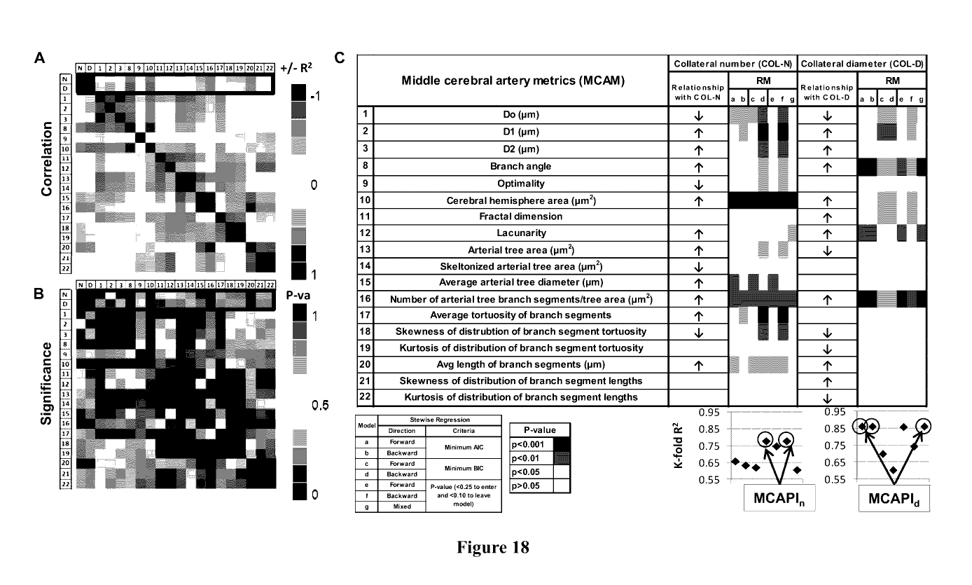

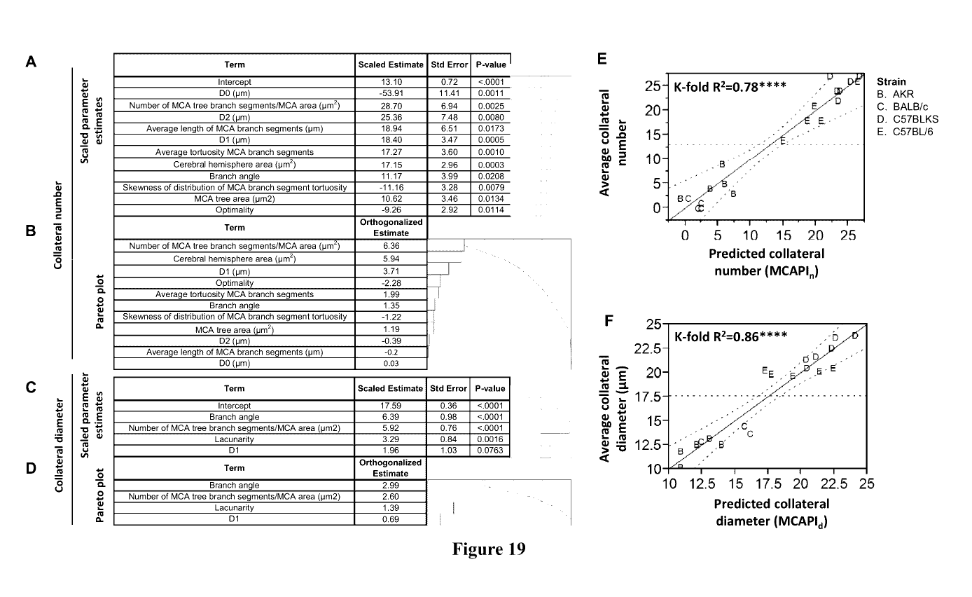

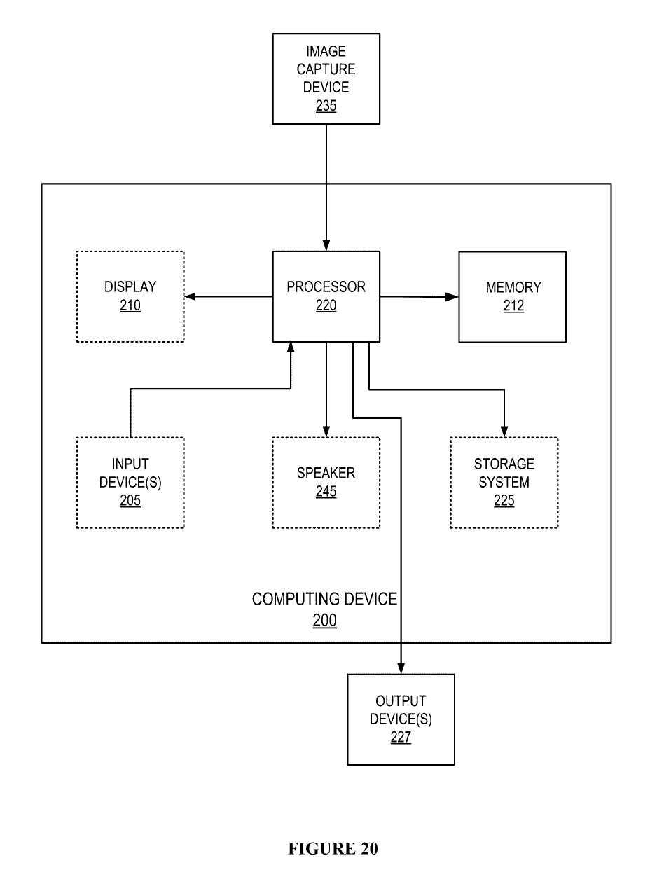

Fractal dimension and lacunarity are global, non-Euclidean dimensionless metrics that have been used to define complexity of the retinal vasculature in association studies. In the present study we found that differences in fractal dimension and lacunarity were associated with differences in retinal patterning metrics (RPMs) (FIG. 5B,C; boxes). Thus, our data set offers a unique opportunity to identify the relationship between these global metrics and Euclidean metrics in a complex branching network, i.e., the retinal vasculature. Fractal dimension was not found to be a significant predictor of COL-N and COL-D across most models (FIG. 15), and lacunarity was only moderately predictive of COL-N(FIG. 8B). These findings were likely due to the narrow range of fractal dimension (1.41-1.54) and high covariance of it and lacunarity with other RPMs (FIG. 5B), many of which were more specific features of arterial tree complexity and thus emerged as stronger predictors of COL-N and COL-D. To better characterize global differences in complexity of the retinal artery trees, as measured by FD and Lac, in terms of Euclidean metrics that are more intuitive and visualizable, we examined the association between RPMs and fractal dimension and lacunarity (FIG. 16). We performed forward, backward, and mixed stepwise multivariate regression modeling of fractal dimension and lacunarity versus RPMs using a variety of criteria as detailed in FIG. 15. Average fractal dimension and lacunarity of retinal artery trees (dependent variables) from 80 mice were modeled versus other RPMs (independent variables) that strictly characterize only arterial tree patterning (i.e., we excluded CRVE, AVR, and retinal area) (FIG. 16A). Significance of RPMs for a given model and their strength of association with fractal dimension and lacunarity, as measured by K-fold R², were identified.

Differences in many RPMs defining retinal arterial tree patterning (vessel caliber, branch angle, tortuosity, etc.) were associated with differences in fractal dimension and lacunarity (i.e., K-fold R² was 0.60-0.64 and 0.47-0.49, respectively) (FIG. 16A). When a given RPM was associated with both fractal dimension and lacunarity, it correlated with both metrics in an opposite direction, consistent with the inverse relationship between fractal dimension and lacunarity (FIG. 16A). The only exception to this observation was average arterial tree diameter, which was directly associated with both fractal dimension and lacunarity. To compare relative strength of association of specific RPMs with fractal dimension and lacunarity, parameter estimates from the 2 most predictive models (FIG. 16A, RPM_{FD} and RPM_{Lac}) were obtained and plotted as scaled estimates (i.e., centered by mean and normalized to have identical range) (FIG. 16B,D). In addition, parameter estimates were standardized to have equal variances, orthogonalized to be uncorrelated, and plotted—in descending order of scaled estimates—as a pareto plot (FIG. 16C,E). The scaled estimates show the relative extent of change in fractal dimension and lacunarity as a specific RPM is varied from the lowest to highest value in the population of mice.

RPMs in descending order of “explanatory power” for fractal dimension and lacunarity. Plots of predicted (i.e. expected) fractal dimension and lacunarity based on models from strongly correlated and explanatory RPMs, along with K-fold R², reveals the spread of data and the strength of correlation (FIG. 16F,G; ****P<0.0001). In addition, segmented arterial trees from two retinas in our study with the widest difference in fractal dimension (1.41 vs. 1.54) and lacunarity (27.0 vs. 13.7) were also compared to better visualize differences in arterial tree patterning in the context of respective differences in RPMs (FIG. 16H,I).

The pareto plot (FIG. 16D) and comparison of retinas (FIG. 16H,I) shows that fractal dimension is disproportionately sensitive to changes in caliber of the central artery (CRAE) in comparison to other RPMs. In general, mice with retinal arterial trees with a larger CRAE, shorter and greater proportion of shorter branch segment lengths (as measured by average length of branch segments and skewness of distribution of branch segment lengths), more tortuous branch segments and a wider distribution of branch segment tortuosity (as measured by average tortuosity of branch segments and kurtosis of distribution of branch segment tortuosity) tend to have a higher FD and lower lacunarity. Higher FD (FIG. 16A,B,I) is also associated with greater extent of coverage of arterial tree (as measured by skeletonized arterial tree area). Other associated differences in RPMs are less visually appreciable, consistent with their relatively lower rank on the pareto plots (FIG. 16B,C,E); for example, low lacunarity is also associated with a lower branch density (as measured by number of arterial tree branch segments/tree area) and a greater proportion of branch segments with a similar length (as measured by kurtosis of distribution of branch segment lengths). As found across all significant models (FIG. 16A), a high fractal dimension and lacunarity were both associated with larger vessel caliber (as measured by average arterial tree diameter). RPMs had a lower strength of association with lacunarity in comparison to fractal dimension (FIG. 16F,G) (K-fold R² 0.49 vs. 0.64), suggesting that, in the context of our study, lacunarity captures additional information on arterial tree patterning beyond the most descriptive RPMs.

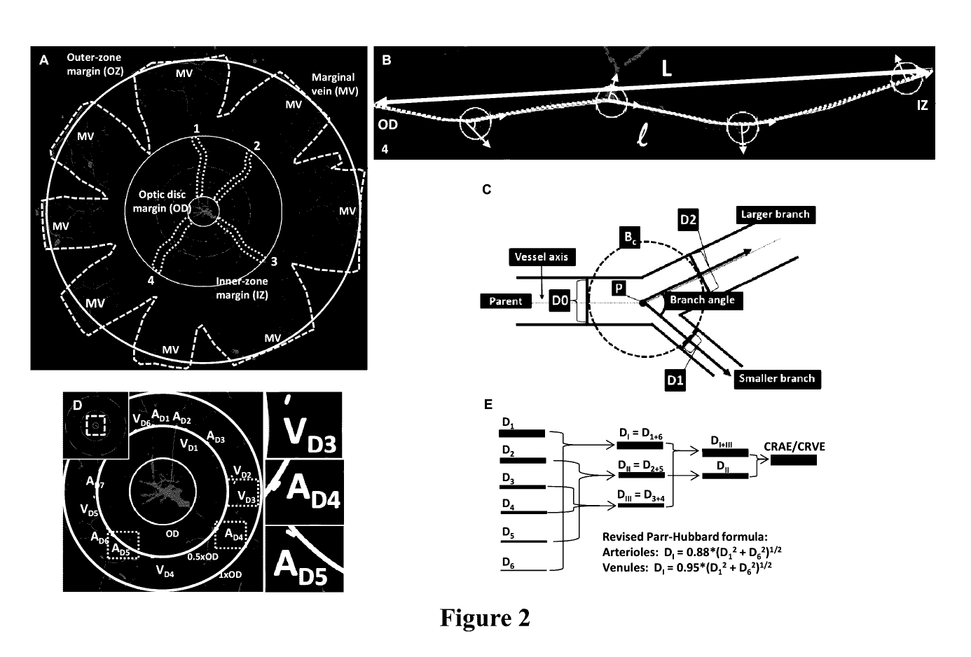

FIG. 17. Both retinal patterning metrics (RPMs) and middle-cerebral artery patterning metrics (MCAM) vary with genetic background, but only half showed significant or suggestive correlations with each other). A subset of 18 MCAMs analogous to patterning metrics derived from the retina (RPMs) was measured in a subset of 21 mice belonging to 4 strains (BALB/c, C57BLKS, AKR, and C57BL/6). Using definitions and methods previously detailed for the derivation of RPMs (FIGS. 2, 4), inner-zone metrics (RPMs/MCAMs 1-3, 8, 9) were measured on the 1^{st} order of the MCA trunk and extended zone metrics (RPMs/MCAMs 11-22) were derived from segmented MCA trees (FIG. 19). Cerebral hemisphere area, the area of the hemisphere supplied by the MCA, is analogous to retinal area (RPM/MCAM 10) supplied by the retinal arterial tree and was measured (FIG. 19). Bivariate regression (one-way ANOVA adjusted R² and p-value) of COL-N, COL-D, and MCAMs versus mouse strain shows that COL-N, COL-D and at least 9 out of 18 MCAMs vary with genetic background (dashed line, cutoff for adjusted R² of >0.35, p=0.0001-0.13), comparable to the 10 out of 22 metrics found to strongly vary with genetic background in the retina (FIG. 5). Black boxes reflect relative strength of correlation. These data suggest that similar to its contribution to the variation in COL-N, COL-D and RPMs, genetic background also plays a significant role in specifying variation in features of the MCA tree, such as vessel caliber, branch angle, fractal dimension, and MCA tree area. Optimality of the MCA tree did not vary with genetic background as it did in the retina, and unlike in the retina, area of the skeletonized MCA tree and cerebral hemisphere and number of arterial tree branch segments per unit tree area showed strong variation with genetic background. Bivariate regression of MCAMs versus analogous RPMs (Bivariate ANOVA adjusted R² and p-value) shows little to no correlation (adjusted R² of 0.02-0.21), with only fractal dimension showing some degree of correlation (adjusted R² of 0.21, p=0.02). The low MCAM-RPM correlation may be attributable to low overall n-size (22), measurement of only 1 MCA tree versus 2-3 trees in the retina, truly flat-mounted 2D measurement in retina versus 3D angular view of the MCA, possible lack of true analogy between MCAMs and RPMs, and reasons related to different times of formation.

FIG. 18. Middle cerebral artery patterning metrics (MCAMs) predict pial collateral number (COL-N) and diameter (COL-D).). Multivariate correlation matrices (A and B) of COL-N(N), COL-D (D) and MCA patterning metrics (MCAMs 1-3, 8-22) show significant covariance, similar to RPMs (FIG. 5); matrices show direction and strength (+/− adjusted R² (A)), and significance (p-value, (B)) of covariance. Highlighted regions of matrices (A,B; boxes) show that a number of MCAMs (2, 8, 10, 15, 16, 20) also vary strongly with COL-N and COL-D, suggesting that they may also predict collateral extent. Thus, similar to methods detailed for RPMs (FIG. 15), forward, backward, and mixed direction stepwise multivariate regression of COL-N and COL-D versus MCAMs was performed using different stopping rules—minimum AIC, minimum BIC, and p-value cutoffs (p<0.25 for RPM to enter and >0.10 to leave model). Predictive performance across all models as determined by K-fold R² calculated from leave-one-out cross validation was strong (0.61-0.78 for COL-N and 0.60-0.86 for COL-D) and confirmed our hypothesis that similar to features of retinal patterning, features of MCA tree patterning strongly associate with and can predict COL-N and COL-D. Directionality, strength, and significance of correlation of MCAMs with COL-N and COL-D across all models is displayed and highlights a select few MCAMs that remained significantly predictive for either COL-N or—D throughout all models; thus, MCA trees with greater branching density (MCAM 16), at wider branch angles (MCAM 8), with smaller parent (D0) but larger daughter vessel calibers (D1, D2) (MCAMs 1-3), supplying larger cerebral hemispheres (MCAM 10) were associated with a greater number of collaterals. The most predictive models based on highest K-fold R² was used to calculate an “MCA predictor index” for COL-N and COL-D similar to the retinal predictor index (MCAPIₙ and MCAPI_{d}, respectively) to further examine and determine comparative predictive ability of individual features of MCA patterning (FIG. 19).

FIG. 19. Among the most predictive MCAMs, average number of MCA tree branch segments per unit MCA area (i.e., MCA branching density) contributes the most, statistically, to predicting collateral number and diameter (COL-N and COL-D). MCA trees with larger branch angle, larger caliber of branching vessels (D1, D2), and larger cerebral hemisphere areas tend to have greater collateral extent (COL-N and/or COL-D). To compare the relative predictive power of the MCAMs, parameter estimates from the 2 most predictive models for MCAPIₙ and MCAPI_{d} (FIG. 18) were obtained and plotted as scaled estimates (i.e. centered by mean and normalized to have identical range) (A,C); in addition, the parameter estimates were standardized to have equal variances, orthogonalized to be uncorrelated, and plotted—in descending order of scaled estimates—as a pareto plot (B,D). The scaled estimates show the relative extent of change in COL-N or COL-D as a specific MCAM is varied from the lowest to highest value in the population of mice. The pareto plot accounts for covariance and extent of variability of an RPM in the mouse population to estimate and arrange the MCAMs in descending order of relative “explanatory power.” Plots of predicted COL-N and COL-D versus MCAPIₙ and MCAPI_{d}, along with K-fold R² reveal the spread of data and the strength of correlation (E,F). ****P<0.0001. Therefore, mice with greater MCA tree branching density, larger branch diameter at bifurcations (D1), and larger branch angles tend to have greater collateral extent (COL-N and COL-D). Mice with relatively larger cerebral hemisphere areas also tend to have greater COL-N.

FIG. 20 is a block diagram of a system including a computing device in accordance with embodiments of the present disclosure.

### SUMMARY OF THE INVENTION

In one aspect, the present invention provides a method of determining a retinal predictor index (RPI) for a tissue of interest of a subject, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAB), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein the RPIₙ corresponds to and/or predicts the collateral number in the tissue of interest and the RPI_{d} corresponds to and/or predicts the average collateral diameter in the tissue of interest.

In a further aspect, the present invention provides a method of identifying the likelihood of poor stroke prognosis in a subject in need thereof, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as having an increased likelihood of poor pial collaterals and poor stroke prognosis and a RPI of the subject that is greater than or equal to a threshold RPI identifies the subject as having an increased likelihood of good pial collaterals and good stroke prognosis.

Also provided herein is a method of identifying the likelihood of poor prognosis in a subject with occlusion or narrowing of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as having an increased likelihood of poor collaterals in the tissue supplied by the occluded or narrowed artery and/or its branches and poor prognosis and a RPI of the subject that is greater than or equal to a threshold RPI identifies the subject as having an increased likelihood of good collaterals in the tissue supplied by the occluded or narrowed artery and/or its branches and good prognosis.

Furthermore, the present invention provides a method of guiding medical treatment of a subject having acute or chronic occlusion or narrowing of an artery and/or its branches and/or having a disease, disturbance and/or pathological condition of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI that is less than a threshold RPI identifies the subject as unsuitable for intra-arterial thrombolytic treatment and an RPI that is greater than a threshold RPI identifies the subject as suitable for intra-arterial thrombolytic treatment.

In an additional aspect, the present invention provides a method of guiding surgical treatment of a subject having acute or chronic occlusion or narrowing of an artery and/or its branches, and/or a disease, disturbance and/or pathological condition of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as unsuitable for mechanical thrombectomy and a RPI of the subject that is greater than a threshold RPI identifies a subject as suitable for mechanical thrombectomy.

Another aspect of this invention includes a method of guiding surgical treatment of a subject having acute or chronic occlusion or narrowing of an artery and/or its branches, and/or a disease, disturbance and/or pathological condition of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as suitable for stent placement and a RPI of the subject that is greater than a threshold RPI identifies the subject as unsuitable for stent placement.

Additionally provided is a method of guiding clinical decision-making for a subject undergoing a procedure involving occlusion of an artery and/or its primary branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI that is greater than a threshold RPI identifies the subject as suitable for a particular course of clinical treatment and an RPI less than a threshold RPI identifies the subject as suitable for a different course of clinical treatment.

The present invention further provides a method of guiding surgical treatment of a subject having an aortic aneurysm, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as suitable for a given type of aortic aneurysm repair and a RPI of the subject that is greater than a threshold RPI identifies the subject as unsuitable for the given type of aortic aneurysm repair.

In additional aspects, the present invention provides a method of producing a retinal predictor index (RPI) nomogram, comprising the steps of: a) obtaining an image of the vascular architecture of the retinal circulation from each subject in a population of subjects; b) determining for each image obtained from each subject in the population of (a), a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, 9) lacunarity, 10) fractal dimension, 11) arterial tree area, 12) skeletonized arterial tree area, 13) average arterial tree diameter, 14) number of arterial tree branch segments/tree area, 15) tortuosity index (inner zone), 16) skewness of distribution of branch segment tortuosity, 17) kurtosis of distribution of branch segment tortuosity, 18) average length of branch segments, 19) skewness of distribution of branch segment lengths, and 20) central retinal artery-to-vein ratio (AVR); c) identifying first key metrics of the patterning metrics of (b) for calculating a retinal predictor index n (RPIₙ) for each subject; d) identifying second key metrics of the patterning metrics of (b) for calculating a retinal predictor index d (RPI_{d}) for each subject; e) calculating, based on the values of the first key metrics, a retinal predictor index n (RPIₙ) for each subject; f) calculating, based on the values of the second key metrics, a retinal predictor index d (RPI_{d}) for each subject; g) calculating a retinal predictor index (RPI) for each subject that is a function based on the RPIₙ and RPI_{d} of each subject; h) determining collateral blood flow for each subject; and i) mathematically and graphically identifying the relationship between the RPI and collateral blood flow for each subject in the population in a format that establishes quintiles for the population, thereby producing the RPI nomogram.

Also provided herein is a retinal predictor index (RPI) nomogram produced by the methods of this invention.

Further provided herein is a method of identifying the likelihood of poor stroke prognosis in a subject in need thereof, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as having an increased likelihood of poor pial collaterals and poor stroke prognosis, and an RPI of the subject that is within the third quintile of the nomogram of this invention identifies the subject as having an increased likelihood of intermediate pial collaterals and intermediate stroke prognosis, and an RPI of the subject that is within the fourth or fifth quintile of the nomogram of this invention identifies the subject as having an increased likelihood of good pial collaterals and good stroke prognosis.

Also provided herein is a method of identifying the likelihood of poor prognosis in a subject with occlusion or narrowing of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as having an increased likelihood of poor pial collaterals and poor prognosis, and an RPI of the subject that is within the third quintile of the nomogram of this invention identifies the subject as having an increased likelihood of intermediate pial collaterals and intermediate prognosis, and an RPI of the subject that is within the fourth or fifth quintile of the nomogram of this invention identifies the subject as having an increased likelihood of good pial collaterals and good prognosis.

In additional aspects of this invention, a method is provided of guiding medical treatment of a subject having acute or chronic occlusion or narrowing of an artery and/or its branches and/or having a disease, disturbance and/or pathological condition of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unsuitable for intra-arterial thrombolytic treatment, and an RPI of the subject that is within the third quintile of the nomogram of this invention identifies the subject as moderately suitable for intra-arterial thrombolytic treatment, and an RPI of the subject that is within the fourth or fifth quintile of the nomogram of this invention identifies the subject as being very suitable for intra-arterial thrombolytic treatment.

Another aspect of this invention provides a method of guiding surgical treatment of a subject having acute or chronic occlusion or narrowing of an artery and its branches, and/or a disease, disturbance and/or pathological condition of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unsuitable for mechanical thrombectomy, and an RPI of the subject that is within the third quintile of the nomogram of this invention identifies the subject as moderately suitable for mechanical thrombectomy, and an RPI of the subject that is within the fourth or fifth quintile of the nomogram of this invention identifies the subject as being very suitable for mechanical thrombectomy.

Another aspect of this invention provides a method of guiding surgical treatment of a subject having acute or chronic occlusion or narrowing of an artery and its branches, and/or a disease, disturbance and/or pathological condition of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unsuitable for stent placement, and an RPI of the subject that is within the third quintile of the nomogram of this invention identifies the subject as moderately suitable for stent placement, and an RPI of the subject that is within the fourth or fifth quintile of the nomogram of this invention identifies the subject as being very suitable for stent placement.

An additional aspect of this invention provides a method of guiding clinical decision-making for a subject undergoing a procedure involving occlusion of an artery and/or its primary branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unsuitable for a particular course of clinical treatment, and an RPI of the subject that is within the third quintile of the nomogram of this invention identifies the subject as moderately suitable for a particular course of clinical treatment, and an RPI of the subject that is within the fourth or fifth quintile of the nomogram of this invention identifies the subject as being very suitable for a particular course of clinical treatment.

Further provided herein is a method of guiding surgical treatment of a subject having an aortic aneurysm, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on ones of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as suitable for a given type of aneurysm repair, and an RPI of the subject that is within the third quintile of the nomogram of this invention identifies the subject as being moderately suitable for a given type of aneurysm repair, and an RPI of the subject that is within the fourth or fifth quintile of the nomogram of this invention identifies the subject as being unsuitable for a given type of aneurysm repair.

In an additional aspect, the present invention provides a computer program product, comprising: a non-transitory computer readable storage medium storing computer readable program code that, when executed by a processor of an electronic device, causes the processor to perform operations comprising: receiving a retinal image that corresponds to a subject and that is generated using an optical device; extracting, binarizing and segmenting one or more of a plurality of retinal artery trees identified in the retinal image; estimating a plurality of retinal patterning metrics corresponding to the retinal image; calculating a retinal predictor index n (RPIₙ) that corresponds to/predicts the number of the collaterals in a tissue of interest; calculating a retinal predictor index d (RPI_{d}) that corresponds to/predicts the average diameter of the collaterals in a tissue of interest; calculating an retinal predictor index (RPI) score using the retinal predictor index n (RPIₙ) and the retinal predictor index d (RPI_{d}); and comparing the RPI to a threshold RPI value.

Furthermore, the present invention provides a computer program product, comprising: a non-transitory computer readable storage medium storing computer readable program code that, when executed by a processor of an electronic device, causes the processor to perform operations described in the methods of this invention.

In yet another aspect, the present invention provides an electronic device comprising: a user interface; a processor; and a memory coupled to the processor and comprising computer readable program code that when executed by the processor causes the processor to perform operations comprising: receiving a retinal image that corresponds to a subject and that is generated using an optical device; extracting, binarizing and segmenting one or more of a plurality of retinal artery trees identified in the retinal image; estimating a plurality of retinal patterning metrics corresponding to the retinal image; calculating a retinal predictor index n (RPIₙ) that corresponds to and/or predicts the collateral number in a tissue of interest; calculating a retinal predictor index d (RPI_{d}) that corresponds to and/or predicts the average collateral diameter in a tissue of interest; calculating a retinal predictor index (RPI) score using the retinal predictor n index (RPIₙ) and the retinal predictor index d (RPI_{d}); and comparing the RPI to a threshold RPI value.

Also provided herein is an electronic device comprising: a user interface; a processor; and a memory coupled to the processor and comprising computer readable program code that when executed by the processor causes the processor to perform operations described in the methods of this invention.

Further provided herein is a system comprising: a retinal image capture device that is configured to capture image data corresponding to vascular architecture of a subject's retinal circulation; a user interface; a processor; and a memory coupled to the processor and comprising computer readable program code that when executed by the processor causes the processor to perform operations described in the methods of this invention.

### DETAILED DESCRIPTION OF THE INVENTION

For the purposes of promoting an understanding of the principles of the present invention, reference will now be made to particular embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the invention relates.

The present invention is based on the unexpected discovery that a subject's pattern of vascularization of the inner retina can be used to predict collateral number and average diameter in other tissue of the subject, which can be used in healthy subjects to predict risk of poor outcome should an occlusive event or disease occur or in treating and/or prognosing subjects having an occlusion or narrowing of an artery and/or its branches. Accordingly, in one embodiment, the present invention provides a method of determining a retinal predictor index (RPI) for a tissue of interest of a subject, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein the RPIₙ corresponds to and/or predicts the collateral number in a tissue of interest and the RPI_{d} corresponds to and/or predicts the average collateral diameter in a tissue of interest.

In various embodiments of this invention, the tissue of interest can be tissue from the brain, spinal cord, heart, lung, an abdominal organ, upper extremity, lower extremity, skin, skeletal muscle, bone and any combination thereof.

The present invention additionally provides a method of identifying the likelihood of poor stroke prognosis in a subject in need thereof, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as having an increased likelihood of poor pial collaterals and poor stroke prognosis and a RPI of the subject that is greater than or equal to the threshold RPI identifies the subject as having an increased likelihood of good pial collaterals and good stroke prognosis.

As used herein, “poor stroke prognosis” means that for the particular location of the occluded vessel(s) and amount of tissue perfused by the vessel(s), plus the elapsed time from onset of occlusion to completion of evaluation to determine the above location and amount, the likelihood of significant improvement of function is poor.

As used herein, “good stroke prognosis” means that for the particular location of the occluded vessel(s) and amount of tissue perfused by the vessel(s), plus the elapsed time from onset of occlusion to completion of evaluation to determine the above location and amount, the likelihood of significant improvement of function is good.

As used herein, “retinal area” is a retinal patterning metric (RPM) that describes the area of the retinal tissue perfused by the inner retinal circulation.

As used herein, “vessel diameter D0” is a RPM that describes the parent vessel giving rise at a bifurcation to two daughter vessels.

As used herein, “vessel diameter D2” is a RPM that describes the daughter vessel of D0 with the larger diameter.

As used herein, “optimality” is a RPM that describes a measure of equitability of distribution of flow from parent to daughter vessels.

As used herein, “branch angle” is a RPM that describes the angle between daughter vessels that bisects D0.

As used herein, “central retinal artery equivalent (CRAE)” is a RPI that describes an estimation of the central retinal artery diameter.

As used herein, “average length of branch segments” is a RPI that describes the average scalar length, l, of all branch segments.

As used herein, “kurtosis of distribution of branch segment lengths” is a RPI that describes the peakedness (or flatness) of distribution of branch segment length; higher kurtosis results from a greater proportion of segments with similar length centered closer to the average.

As used herein, “lacunarity” is a RPM that describes a dimensionless measure of vessel complexity closely related to fractal dimension.

As used herein, “retinal predictor index for collateral number (RPIₙ)” is a number that predicts collateral number and is specified by a formula derived from multivariate modeling of RPMs.

As used herein, “retinal predictor index for average collateral diameter (RPI_{d})” is a number that predicts average collateral diameter and is specified by a formula derived from multivariate modeling of RPMs.

As used herein, a “retinal predictor index (RPI)” is the product (and/or other mathematical function) of RPIₙ and RPI_{d} to yield a single number encompassing both RPIₙ and RPI_{d}.

As used herein, a “threshold RPI” describes a RPI derived from a population of individuals that allows predicting whether the given individual has poor collaterals (less than the threshold RPI) versus good collaterals (greater than or equal to the threshold RPI). It is determined by a mathematical function (e.g., see also “nomogram” below) derived from a test population of individuals used to establish the relationship between RPI and collateral score or status as determined by neuroimaging or other method of measurement of collateral-dependent blood flow or collateral number and diameter in the target tissue (eg, brain, heart, lower extremity).

As used herein, the terms “good pial collaterals” or “good collaterals” describe an individual with a calculated RPI at or above a threshold RPI.

As used herein, the terms “poor pial collaterals” or “poor collaterals” describe an individual with a calculated RPI below a threshold RPI.

Also provided herein is a method of identifying the likelihood of poor prognosis in a subject with occlusion or narrowing of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as having an increased likelihood of poor collaterals in the tissue supplied by the occluded or narrowed artery and/or its branches and poor prognosis and a RPI of the subject that is greater than or equal to the threshold RPI identifies the subject as having an increased likelihood of good collaterals in the tissue supplied by the occluded or narrowed artery and/or its branches and good prognosis.

Further provided herein is a method of guiding medical treatment of a subject having acute or chronic occlusion or narrowing of an artery and/or its branches and/or having a disease, disturbance and/or pathological condition of an artery and/or its branches that causes occlusion or narrowing, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI that is less than a threshold RPI identifies the subject as likely to benefit significantly from intra-arterial thrombolytic treatment and an RPI that is greater than or equal to the threshold RPI identifies the subject as not likely to benefit significantly from thrombolytic treatment or other revascularization therapies.

Non-limiting examples of a disease, disturbance and/or pathological condition of an artery and/or its branches include embolic occlusion or stenosis, thrombotic occlusion or stenosis, athero-occlusion or stenosis, coarctation, dissection-induced occlusion or stenosis, and vascular wall hypertrophic occlusion or stenosis.

Non-limiting examples of other medical treatments that can be chosen based on whether a subject has an RPI either less than or greater than a threshold, that classifies them as unsuitable versus suitable for a given treatment include treatments that augment positive remodeling of pre-existing (native) collaterals (e.g., TRP-V4 agonists); that induce and/or augment formation of new collaterals (e.g., CCR2⁺ or CX₃CR1⁺ bone marrow derived cells); and/or that augment blood flow across the collaterals (e.g., tissue-specific vasodilators, administering an agent that increases blood pressure), as would be known to one of skill in the art.

Also provided herein is a method of guiding surgical treatment of a subject having acute or chronic occlusion or narrowing of an artery and/or its branches, and/or a disease, disturbance and/or pathological condition of an artery and/or its branches that causes occlusion or narrowing, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as unlikely to significantly benefit from mechanical thrombectomy or clot disruption and a RPI of the subject that is greater than or equal to the threshold RPI identifies the subject as likely to significantly benefit from mechanical thrombectomy or clot disruption.

The present invention further provides a method of guiding surgical treatment of a subject having acute or chronic occlusion or narrowing of an artery and/or its branches, and/or a disease, disturbance and/or pathological condition of an artery and/or its branches that causes occlusion or narrowing, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as unlikely to significantly benefit from stent placement and a RPI of the subject that is greater than or equal to the threshold RPI identifies the subject as likely to significantly benefit from angioplasty and/or stent placement.

Additionally provided is a method of guiding clinical decision-making for a subject undergoing a procedure involving occlusion or narrowing of an artery and/or its primary branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI that is greater than or equal to a threshold RPI identifies the subject as likely to significantly benefit from a particular course of clinical treatment (e.g., treatments that would be appropriate for an individual having good collateral blood flow to the area at risk, i.e., as defined by the site of occlusion) and a RPI that is less than the threshold RPI identifies the subject as likely to significantly benefit from a different course of clinical treatment (e.g., treatments that would be appropriate for an individual having poor collateral blood flow to the area at risk).

As used herein, “area at risk” refers to the tissue volume normally perfused by the arterial tree downstream from the point that is currently narrowed (stenosed) or occluded (blocked) in the affected individual.

Additional non-limiting examples of courses of treatment would include a decision whether to administer versus not administer a course of medical and/or surgical treatment to open the occluded/narrowed vessel, as well as a no versus yes decision regarding whether to administer a course of medical treatment to augment positive remodeling of pre-existing (native) collaterals, and/or induce or augment formation of new collaterals, and/or augment blood flow across the collaterals as described herein.

The present invention further provides a method of guiding surgical treatment of a subject having an aneurysm of the aorta or other artery, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is less than a threshold RPI identifies the subject as likely to significantly benefit from a given type of repair of an aneurysm of the aorta or other artery and a RPI of the subject that is greater than or equal to the threshold RPI identifies the subject as likely to significantly benefit from a different type of aneurysm repair. For example, a surgical procedure to repair an aorta aneurysm that would occlude a branch(s) off of the aorta would be indicated for an individual with an RPI greater than or equal to the threshold RPI, whereas a different surgical procedure to repair the aneurysm that would provide a different sequence or amount of branch occlusion(s) would be indicated for an individual with an RPI less that the threshold RPI.

In some embodiments of the invention, the treatment can comprise, e.g., intestinal resection, repair of aortic and arterial aneurysms, and/or cannulation of an artery.

The present invention also contemplates the use of a nomogram for carrying out the methods of this invention. Accordingly, in one embodiment, the present invention provides a method of producing a retinal predictor index (RPI) nomogram, comprising the steps of: a) obtaining an image of the vascular architecture of the retinal circulation from each subject in a population of subjects; b) determining for each image obtained in (a), a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity, 10) fractal dimension, 11) arterial tree area, 12) skeletonized arterial tree area, 13) average arterial tree diameter, 14) number of arterial tree branch segments/tree area, 15) tortuosity index (inner zone), 16) skewness of distribution of branch segment tortuosity, 17) kurtosis of distribution of branch segment tortuosity, 18) average length of branch segments, 19) skewness of distribution of branch segment lengths, and 20) central retinal artery-to-vein ratio (AVR); c) identifying first key metrics among the patterning metrics of (b) for calculating a retinal predictor index n (RPIₙ) for each subject; d) identifying second key metrics among the patterning metrics of (b) for calculating a retinal predictor index d (RPI_{d}) for each subject; e) calculating, based on the values of one or more of the first key metrics, a retinal predictor index n (RPIₙ) for each subject; f) calculating, based on one or more of the values of the second key metrics, a retinal predictor index d (RPI_{d}) for each subject; g) calculating a retinal predictor index (RPI) for each subject that is a function based on the RPIₙ and RPI_{d} of each subject; h) determining collateral blood flow for each subject or a surrogate of this (e.g., collateral number and/or diameter and/or collateral score from neuroimaging or other imaging modalities); and i) mathematically and graphically identifying the relationship between the RPI and collateral blood flow (or surrogate thereof) for each subject in the population in a format that establishes quintiles for the population, thereby producing a RPI nomogram.

As used herein, a “retinal predictor index (RPI) nomogram” describes the mathematical function relating RIP to collateral blood flow or a surrogate thereof for a population of individuals.

As used herein, “determining collateral blood flow” means obtaining a measure of collateral-dependent blood flow or a surrogate thereof (e.g., collateral number and/or diameter, and/or collateral score on neuroimaging; e.g., coronary collateral flow indexₚ; e.g., number and/or diameter and/or area-length density of collaterals in a tissue as determined by angiography).

As used herein, mathematically and graphically identifying the relationship between the RPI and collateral blood flow or a surrogate thereof in a population of individual so as to establish quintiles for the population means determining the population-wise function relating RPI to collateral blood flow or a surrogate thereof by analyzing the individuals in the population to derive that average relationship.

Also provided herein is a retinal predictor index (RPI) nomogram produced according to the method of this invention.

In further embodiments, the present invention provides a method of identifying the likelihood of poor stroke prognosis in a subject in need thereof, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as having an increased likelihood of poor pial collaterals and poor stroke prognosis, and an RPI of the subject that is within the third quintile of the nomogram of this invention identifies the subject as having an increased likelihood of intermediate pial collaterals and intermediate stroke prognosis, and an RPI of the subject that is within the fourth or fifth quintile of the nomogram of this invention identifies the subject as having an increased likelihood of good pial collaterals and good stroke prognosis.

As used herein, “intermediate pial collaterals” means an individual whose RPI falls within the 3^{rd} quintile of the population nomogram.

As used herein, “intermediate stroke prognosis” means a subject with a prognosis between good and poor.

Also provided herein is a method of identifying the likelihood of poor prognosis in a subject with occlusion or narrowing of an artery and/or its branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as having an increased likelihood of poor pial collaterals and poor prognosis, and an RPI of the subject that is within the third quintile of the nomogram of this invention identifies the subject as having an increased likelihood of intermediate pial collaterals and intermediate prognosis, and an RPI of the subject that is within the fourth or fifth quintile of the nomogram of this invention identifies the subject as having an increased likelihood of good pial collaterals and good prognosis.

As used herein, “poor prognosis in a subject with occlusion or narrowing of an artery and/or its branches” means a subject likely to sustain significant tissue injury.

As used herein, “intermediate prognosis in a subject with occlusion or narrowing of an artery and/or its branches” means a subject likely to sustain moderate tissue injury.

As used herein, “good prognosis in a subject with occlusion or narrowing of an artery and/or its branches” means a subject likely to sustain minimal to no tissue injury.

Further provided herein is a method of guiding medical treatment of a subject having acute or chronic occlusion or narrowing of an artery and/or its branches and/or having a disease, disturbance and/or pathological condition of an artery and/or its branches that causes occlusion or narrowing, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unlikely to benefit significantly from intra-arterial thrombolytic treatment, and an RPI of the subject that is within the third quintile of the nomogram of this invention identifies the subject as likely to moderately benefit from intra-arterial thrombolytic treatment, and an RPI of the subject that is within the fourth or fifth quintile of the nomogram of this invention identifies the subject as likely to significantly benefit from intra-arterial thrombolytic treatment.

As used herein, “unlikely to significantly benefit from intra-arterial thrombolytic treatment” means an individual that is unlikely to sustain a significantly smaller amount of tissue injury as a result of the treatment.

As used herein, “likely to moderately benefit from intra-arterial thrombolytic treatment” means an individual that is likely to sustain a moderate reduction in the amount of tissue injury as a result of the treatment.

As used herein, “likely to significantly benefit from intra-arterial thrombolytic treatment” means an individual likely to sustain a significant reduction in the amount of tissue injury as a result of the treatment.

Additionally provided herein is a method of guiding surgical treatment of a subject having acute or chronic occlusion or narrowing of an artery and its branches, and/or a disease, disturbance and/or pathological condition of an artery and/or its branches that causes occlusion or narrowing, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unlikely to significantly benefit from mechanical thrombectomy or clot disruption, and an RPI of the subject that is within the third quintile of the nomogram of this invention identifies the subject as likely to moderately benefit from mechanical thrombectomy or clot disruption, and an RPI of the subject that is within the fourth or fifth quintile of the nomogram of this invention identifies the subject as likely to significantly benefit from mechanical thrombectomy or clot disruption.

The present invention further provides a method of guiding surgical treatment of a subject having acute or chronic occlusion or narrowing of an artery and its branches, and/or a disease, disturbance and/or pathological condition of an artery and/or its branches that causes occlusion or narrowing, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unlikely to significantly benefit from stent placement, and an RPI of the subject that is within the third quintile of the nomogram of this invention identifies the subject as likely to moderately benefit from stent placement, and an RPI of the subject that is within the fourth or fifth quintile of the nomogram of this invention identifies the subject as likely to significantly benefit from stent placement and/or angioplasty.

Furthermore, the present invention provides a method of guiding clinical decision-making for a subject undergoing a procedure involving occlusion of an artery and/or its primary branches, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as unlikely to benefit significantly from a particular course of clinical treatment, and an RPI of the subject that is within the third quintile of the nomogram of this invention identifies the subject likely to moderately benefit from a particular course of clinical treatment, and an RPI of the subject that is within the fourth or fifth quintile of the nomogram of this invention identifies the subject as likely to significantly benefit from a particular course of clinical treatment. An example of a clinical course of treatment is whether or not to use one or more of the following: intra-arterial thrombolytic, clot retrieval, clot disruption, stent placement, a particular type of aneurism repair procedure versus an alternative repair procedure, medical treatments or therapies that augment collateral remodeling, new collateral formation, or that augment collateral flow across the current/existing collateral vessels.

In additional embodiments, the present invention provides a method of guiding surgical treatment of a subject having an aortic aneurysm, comprising: a) obtaining an image of the vascular architecture of the subject's retinal circulation; b) determining a value for the following patterning metrics of retinal artery trees in the image: 1) retinal area, 2) vessel diameter D0, 3) vessel diameter D2, 4) optimality, 5) branch angle, 6) central retinal artery equivalent (CRAE), 7) average length of branch segments, 8) kurtosis of distribution of branch segment lengths, and 9) lacunarity; c) calculating, based on one or more of the values of the patterning metrics, a retinal predictor index for collateral number (RPIₙ) and a retinal predictor index for average collateral diameter (RPI_{d}) for the subject; and d) calculating a retinal predictor index (RPI) that is a function based on the RPIₙ and RPI_{d}, wherein a RPI of the subject that is within the first or second quintile of the nomogram of this invention identifies the subject as likely to significantly benefit from a given type of aortic aneurysm repair, wherein a RPI of the subject that is within the third quintile of the nomogram of this invention identifies the subject as likely to significantly benefit from one type of aortic aneurysm repair over another when other clinical factors are taken into consideration, and wherein a RPI of the subject that is within the fourth or fifth quintile of the nomogram for this invention identifies the subject as likely to significantly benefit from a different type of aortic aneurysm repair from that used in individuals with RPIs in the first or second quintiles.

In some embodiments of this invention, the artery can be an intracranial or extracranial cerebral artery and in particular embodiments, the subject can have acute or chronic stroke. In some embodiments of this invention, the artery can be a coronary artery and in particular embodiments, the subject can have acute or chronic myocardial infarction and/or coronary artery disease.

In some embodiments of this invention, the artery can supply in a leg, arm, foot and/or hand of a subject, singly or in any combination. In particular embodiments, the subject can have peripheral artery disease.

In some embodiments, the artery can be the aorta or other artery of the subject. In particular embodiments, the subject can have a disease, disturbance or pathological condition of the aorta wall, e.g., Marfan's syndrome, vasculitis, or aortic atherosclerosis that changes the diameter (intraluminal and/or extralumenal) of the artery.

Any of the methods described above can further comprise the steps of assessing the subject's clinical parameters, medical history and demographics, and factoring them with the subject's RPI to determine a course of medical and/or surgical treatment. Clinical parameters include duration of occlusion, blood pressure, blood glucose, body temperature, results from angiography, CT or MR imaging. Medical history include presence of hypertension, type 1 or 2 diabetes, dyslipidemia, CAD, PAD, other disease/disorders, medications in use. Demographic details include sex, age, race/ethnicity. Factors from each of these three categories can be factored into interpreting the information provided by the subjects RPI. For example, low blood pressure will favor worse symptoms in a patient with acute ischemic stroke despite his/her having an RPI in the fourth or fifth quintile; e.g., presence of hypertension or advanced aging associate with lower collateral score/status, collateral blood flow and surrogates thereof. Accordingly, in some embodiments, the methods of guiding medical treatment, guiding surgical treatment and clinical decision making can include consideration of these additional demographic, medical history and/or clinical parameters.

As a non-limiting example of how the subject's clinical parameters can be factored with the subject's RPI, if the subject is hypotensive and is predicted to have poor collaterals as indicated by the subject's RPI, then medical treatment to raise arterial pressure could be deemed unsuitable, whereas if the subject is hypotensive and is predicted to have good pial collaterals as indicated by the subject's RPI, the treatment to raise arterial pressure could be deemed suitable.

In the methods described above, calculating the retinal predictor index n (RPIₙ) can comprise, consist essentially of or consist of calculating the retinal predictor index n (RPIₙ) for the subject using the vessel diameter D2, the average length of branch segments, the retinal area, the kurtosis of distribution of branch segment lengths, the branch angle, the lacunarity, the optimality, the central retinal artery equivalent (CRAE), and the vessel diameter D0.

In some embodiments, the retinal predictor index n (RPIn) can comprise a sum of: a summative constant j; a product of the vessel diameter D2 and a coefficient a; a product of the average length of branch segments and a coefficient b; a product of the retinal area and a coefficient c; a product of the kurtosis of distribution of branch segment lengths and a coefficient d; a product of the branch angle and a coefficient e; a product of the lacunarity and a coefficient f; a product of the optimality and a coefficient g; a product of the CRAE and a coefficient h; and a product of the vessel diameter D0 and a coefficient k, wherein the summative constant j is in a range of about −4.0 to about 12.0, wherein the coefficient a is in a range of about 2.0 to about 6.0, wherein the coefficient b is in a range of about −1.0 to about 1.0, wherein the coefficient c is in a range of about 1.0*10⁻⁵ to about 1.0*10⁻⁸, wherein the coefficient d is in a range of about −1.0 to about 1.0, wherein the coefficient e is in a range of about 0.10 to about 0.40, wherein the coefficient f is in a range of about 0.25 to about 0.70, wherein the coefficient g is in a range of about −19.0 to about −36.0, wherein the coefficient h is in a range of about 0.05 to about 0.50, and wherein the coefficient k is in a range of about −3.0 to about 3.0.

In particular embodiments of the invention, the summative constant j can be about 4.91±17.2 (standard error of 8.80); the coefficient a can be about 2.91±1.47, (standard error of 0.75); the coefficient b can be about −0.511±0.151, (standard error of 0.08); the coefficient c can be about 1.1*10⁻⁶±4.95e-7, (standard error of 2.52*10-7); the coefficient d can be about −0.268±0.114, (standard error of 0.058); the coefficient e can be about 0.222±0.098, (standard error of 0.050); the coefficient f can be about 0.443±0.265, (standard error of 0.135); the coefficient g can be about −27.3±16.5, (standard error of 8.41); the coefficient h can be about 0.262±0.318, (standard error of 0.161); and the coefficient k can be about −1.96±1.61. (standard error of 0.820).

In the methods described above, calculating the retinal predictor index d (RPI_{d}) can comprises, consist essentially of or consist of calculating the retinal predictor index d (RPI_{d}) for the subject using the vessel diameter D2, the average length of branch segments, the retinal area, the optimality, the kurtosis of distribution of branch segment lengths, the vessel diameter D0, and the branch angle.

In some embodiments, the retinal predictor index d (RPI_{d}) can comprise the sum of: a summative constant m; a product of the vessel diameter D2 and a coefficient n; a product of the average length of branch segments and a coefficient p; a product of the retinal area and a coefficient q; a product of the optimality and a coefficient r; a product of the kurtosis of distribution of branch segment lengths and a coefficient s; a product of the vessel diameter D0 and a coefficient t; and a product of the branch angle and a coefficient u, wherein the summative constant m is in a range of about 10.0 to about 30.0, wherein the coefficient n is in a range of about 0.5 to about 3.5, wherein the coefficient p is in a range of about −0.05 to about −0.40, wherein the coefficient q is in a range of about 5.0*10⁻⁶ to about 5.0*10⁻⁸, wherein the coefficient r is in a range of about −1.0 to about −20.0, wherein the coefficient s is in a range of about −0.005 to about −0.15, wherein the coefficient t is in a range of about −2.5 to about 0.01, and wherein the coefficient u is in a range of about 0.01 to about 0.20.

In particular embodiments of the invention, the summative constant m can be about 20.3±8.51 (standard error of 4.34); the coefficient n can be about 1.790.751 (standard error of 0.383); the coefficient p can be about −0.2290.082 (standard error of 0.042); the coefficient q can be about 5.4*10⁻⁷±2.86e-7 (standard error of 1.46e-7); the coefficient r can be about −11.6±8.41 (standard error of 4.29); the coefficient s can be about −0.0930±0.063 (standard error of 0.032); the coefficient t can be about −1.370.747 (standard error of 0.381); and the coefficient u can be about 0.1030.057 (standard error of 0.029).

In the methods described herein, calculating the RPI can comprise performing a mathematical operation on RPIₙ and RPI_{d}. In some embodiments, the mathematical operation can be multiplication. In some embodiments, the mathematical operation can be addition. In some embodiments the mathematical operation can include multiplication of one or more retinal patterning metrics by a coefficient.

Any of the methods described above can further comprise the steps of determining a value for the retinal patterning metrics: 1) fractal dimension, 2) arterial tree area, 3) skeletonized arterial tree area, 4) average arterial tree diameter, 5) number of arterial tree branch segments/tree area, 6) tortuosity index (inner zone), 7) skewness of distribution of branch segment tortuosity, 8) kurtosis of distribution of branch segment tortuosity, 18) average length of branch segments, 9) skewness of distribution of branch segment lengths, and/or 10) central retinal artery-to-vein ratio (AVR).

Any of the methods described above can further comprise the steps of determining a value for the retinal patterning metrics: 1) Branch lengths distribution points: Branch lengths maximum, 2) Branch lengths distribution points: Branch lengths minimum, 3) Branch lengths distribution points: Branch lengths 25^{th} percentile, 4) Branch lengths distribution points: Branch lengths 75^{th} percentile, 5) Branch lengths distribution points: Branch lengths median, 6) Tortuosity of branches distribution points: Tortuosity maximum, 7) Tortuosity of branches distribution points: Tortuosity minimum, 8) Tortuosity of branches distribution points: Tortuosity 25^{th} percentile, 9) Tortuosity of branches distribution points: Tortuosity 75^{th} percentile, 10) Tortuosity of branches distribution points: Tortuosity median, 11) Average tortuosity of branch segments, 12) Number of bifurcations per tree, 13) Number of trees crossing the optic disc demarcator, 14) Number of trees crossing the inner zone margin, 15) Percent area skeletonized on area canvas used to obtain fractal dimension and lacunarity (e.g., 25×25), 16) Total length based on Image J analyze skeleton plugin, 17) Average diameter, 18) Number of branches, 19) Number of junctions, 20) Number of end-points, 21) Average branch length from calculated total, 22) Average branch length from analyze skeleton plugin based total length, 23) N number of branches, 24) N number of junctions, 25) N number of end-points, 26) Hull span ratio, 27) Fractal dimension from Image J plugin, and/or 28) Lacunarity from Image J plugin.

It is contemplated that for any of the methods of this invention, one or more of the steps or operations described therein can be performed using at least one processor. Accordingly, a further embodiment of the present invention provides a computer program product, comprising: a non-transitory computer readable storage medium storing computer readable program code that, when executed by a processor of an electronic device, causes the processor to perform operations comprising: receiving a retinal image that corresponds to a subject and that is generated using an optical device; extracting, binarizing and segmenting one or more of a plurality of retinal artery trees identified in the retinal image; estimating a plurality of retinal patterning metrics corresponding to the retinal image; calculating a retinal predictor index n (RPIₙ) that corresponds to/predicts the number of the collaterals in a tissue of interest; calculating a retinal predictor index d (RPI_{d}) that corresponds to/predicts the average diameter of the collaterals in a tissue of interest; calculating an retinal predictor index (RPI) score using the retinal predictor index n (RPIₙ) and the retinal predictor index d (RPI_{d}); and comparing the RPI to a threshold RPI value.

The computer program product of this invention can further comprise an operation of identifying a likelihood of poor collaterals thus poor prognosis, or good collaterals thus good prognosis, in a subject with stroke and/or with acute or chronic occlusion and/or narrowing of an artery and/or its branches, and/or with a disease, disturbance or pathological condition of an artery and/or its branches, responsive to comparing the RPI to a threshold RPI value.

In some embodiments, the computer program product of this invention can further comprise an operation of identifying guidance for medical treatment of a subject having acute or chronic occlusion and/or narrowing of an artery and/or its branches, and/or a disease, disturbance and/or pathological condition of an artery, responsive to comparing the RPI to a threshold RPI value.

In some embodiments, the computer program product of this invention can further comprise an operation of identifying guidance for surgical treatment of a subject having acute or chronic occlusion of an artery and/or its branches, and/or a disease, disturbance and/or pathological condition of an artery, responsive to comparing the RPI to a threshold RPI value.

In some embodiments, the computer program product of this invention can further comprising an operation of identifying guidance for clinical decision-making of a subject undergoing a procedure involving occlusion of an artery and/or its branches, responsive to comparing the RPI to a threshold RPI value.

Additionally provided herein is a computer program product, comprising: a non-transitory computer readable storage medium storing computer readable program code that, when executed by a processor of an electronic device, causes the processor to perform operations described in any of the methods of this invention.

Further provided herein is an electronic device comprising: a user interface; a processor; and a memory coupled to the processor and comprising computer readable program code that when executed by the processor causes the processor to perform operations comprising: receiving a retinal image that corresponds to a subject and that is generated using an optical device; extracting, binarizing and segmenting one or more of a plurality of retinal artery trees identified in the retinal image; estimating a plurality of retinal patterning metrics corresponding to the retinal image; calculating a retinal predictor index n (RPIₙ) that corresponds to and/or predicts the collateral number in a tissue of interest; calculating a retinal predictor index d (RPI_{d}) that corresponds to and/or predicts the average collateral diameter in a tissue of interest; calculating a retinal predictor index (RPI) score using the retinal predictor n index (RPIₙ) and the retinal predictor index d (RPI_{d}); and comparing the RPI to a threshold RPI value.

The electronic device of this invention can further comprise an operation of identifying a likelihood of poor or good prognosis in a subject responsive to comparing the RPI to a threshold RPI value.

The electronic device of this invention can further comprise an operation of identifying guidance for medical treatment of a subject having acute or chronic occlusion of an artery and/or its branches, and/or a disease, disturbance and/or pathological condition an artery, responsive to comparing the RPI to a threshold RPI value.

The electronic device of this invention can further comprise an operation of identifying guidance for surgical treatment of a subject having acute or chronic occlusion of an artery and/or its branches, and/or a disease, disturbance and/or pathological condition of an artery, responsive to comparing the RPI to a threshold RPI value.

The electronic device of this invention can further comprise an operation of identifying guidance for clinical decision making of a subject undergoing a procedure involving occlusion of an artery and/or its branches, responsive to comparing the RPI to a threshold RPI value.

The present invention also provides an electronic device comprising: a user interface; a processor; and a memory coupled to the processor and comprising computer readable program code that when executed by the processor causes the processor to perform operations described in any of the methods of this invention.

FIG. 20 illustrates an example image capture device 235 and an example computing device 200 in accordance with some embodiments of the present disclosure. The computing device 200 may receive image data from the image capture device 235. For example, the image data may include an image of the vascular architecture of a subject's retinal circulation. The computing device 200 may use hardware, software implemented with hardware, firmware, tangible computer-readable storage media having instructions stored thereon, or a combination thereof, and may be implemented in one or more computer systems or other processing systems. The computing device 200 may also be a virtualized instance of a computer. As such, the devices and methods described herein may be embodied in any combination of hardware and software.

As shown in FIG. 20, the computing device 200 may include input device(s) 205, such as a keyboard or keypad, a display 210, and a memory 212 that communicate with one or more processors 220 (generally referred to herein as “a processor”). The computing device 200 may further include a storage system 225, and a speaker 245, that also communicate with the processor 220. The memory 212 may include instructions that when executed by the processor 220, perform operations corresponding to any of the methods described herein.

The storage system 225 may include removable and/or fixed non-volatile memory devices (such as but not limited to a hard disk drive, flash memory, and/or like devices that may store computer program instructions and data on computer-readable media), volatile memory devices (such as but not limited to random access memory), as well as virtual storage (such as but not limited to a RAM disk). Although illustrated in separate blocks, the memory 212 and the storage system 225 may be implemented by a same storage medium in some embodiments.

Although not illustrated herein, one or more communication interfaces may be used to transfer information in the form of signals between the computing device 200 and the image capture device, an output device 227 and/or another computer system or a network (e.g., the Internet). The communication interface may include a modem, a network interface (such as an Ethernet card), a communications port, a PCMCIA slot and card, or the like. These components may be conventional components, such as those used in many conventional computing devices, and their functionality, with respect to conventional operations, is generally known to those skilled in the art. Communication infrastructure between the components of FIG. 2 may include one or more device interconnection buses such as Ethernet, Peripheral Component Interconnect (PCI), and the like.

The computing device 200 may transmit values, metrics, image data and/or data providing guidance for clinical decision making to the output device 227. The output device 227 may include a printer, projector, and/or display that may be separate from and/or include the display 205. Although illustrated as separate elements, the computing device 200 may include the image capture device 235 such that a unitary device may be capable of performing operations described herein.

### DEFINITIONS

The terms “a,” “an” and “the” are used herein to refer to one or to more than one (i.e., at least one) of the grammatical object of the article. By way of example, “an element” means at least one element and can include more than one element (e.g., a multiplicity or plurality of elements).

As used herein, the term “and/or” refers to and encompasses any and all possible combinations of one or more of the associated listed items, as well as the lack of combinations when interpreted in the alternative (“or”).

As used herein, the term “about,” when used in reference to a measurable value such as an amount of mass, dose, time, temperature, and the like, is meant to encompass variations of 20%, 10%, 5%, 1%, 0.5%, or even 0.1% of the specified amount.

As used herein, “one or more” can mean one, two, three, four, five, six, seven, eight, nine, ten or more, up to any number.

Unless otherwise defined, all technical terms used herein have the same meaning as commonly understood by one of ordinary skill in the art to which this disclosure belongs.

As used herein, the term “subject” and “patient” are used interchangeably herein and refer to both human and nonhuman animals. A subject of this invention can be any subject that is susceptible to occlusion or narrowing of an artery and/or its branches and/or has had or is having a disease, disturbance and/or pathological condition of an artery and/or its branches and in particular embodiments, the subject of this invention is a human subject.

A “subject in need thereof” or “a subject in need of” is a subject known to have, or is suspected of having or developing occlusion or narrowing of an artery and/or its branches and/or has had or is having a disease, disturbance and/or pathological condition of an artery and/or its branches. In particular embodiments, the subject is in need of, is scheduled for and/or is planning to undergo a procedure involving occlusion of an artery and/or its primary branches

The term “administering” or “administered” as used herein is meant to include topical, parenteral and/or oral administration, all of which are described herein. Parenteral administration includes, without limitation, intravenous, subcutaneous and/or intramuscular administration (e.g., skeletal muscle or cardiac muscle administration). It will be appreciated that the actual method and order of administration will vary according to, inter alia, the particular preparation of compound(s) being utilized, and the particular formulation(s) of the one or more other compounds being utilized. The optimal method and order of administration of the compounds of the invention for a given set of conditions can be ascertained by those skilled in the art using conventional techniques and in view of the information set out herein.

The term “administering” or “administered” also refers, without limitation, to oral, sublingual, buccal, transnasal, transdermal, rectal, intramuscular, intravenous, intraarterial (intracoronary), intraventricular, intrathecal, and subcutaneous routes. In accordance with good clinical practice, the instant compounds can be administered at a dose that will produce effective beneficial effects without causing undue harmful or untoward side effects, i.e., the benefits associated with administration outweigh the detrimental effects.

Also as used herein, the terms “treat,” “treating” or “treatment” refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, and/or change in clinical parameters of the condition, disorder, disease or illness, etc., as would be well known in the art.

Additionally as used herein, the terms “prevent,” preventing” or “prevention” refer to any type of action that results in the absence, avoidance and/or delay of the onset and/or progression of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

An “effective amount” or “therapeutically effective amount” refers to an amount of a compound or composition of this invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

As used herein, the term “ameliorate” refers to the ability to make better, or more tolerable, a condition such as occlusion or narrowing of an artery and/or its branches and/or a disease, disturbance and/or pathological condition of an artery and/or its branches. In some embodiments, the term “prevent” refers to the ability to keep a condition such as occlusion or narrowing of an artery and/or its branches and/or a disease, disturbance and/or pathological condition of an artery and/or its branches from happening or existing as well as to diminish or delay onset. In some embodiments, the term “treating” refers to the caring for, or dealing with, a condition such as occlusion or narrowing of an artery and/or its branches and/or a disease, disturbance and/or pathological condition of an artery and/or its branches medically and/or surgically.

Pharmaceutical compositions may be prepared as medicaments to be administered in any method suitable for the subject's condition, for example, orally, parenterally (including subcutaneous, intramuscular, and intravenous), rectally, transdermally, buccally, or nasally, or may be delivered directly to the heart by injection and/or catheter, or may be delivered to the eye as a liquid solution.

“Pharmaceutically acceptable,” as used herein, means a material that is not biologically or otherwise undesirable, i.e., the material may be administered to a subject along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; latest edition). Exemplary pharmaceutically acceptable carriers for the compositions of this invention include, but are not limited to, sterile pyrogen-free water and sterile pyrogen-free physiological saline solution, as well as other carriers suitable for injection into and/or delivery to a subject of this invention, particularly a human subject, as would be well known in the art.

In some embodiments, a unique form of parenteral administration is via direct access to the coronary circulation, added to cardioplegia solutions routinely used during cardiac surgery. Such delivery can follow an antegrade route (via the aortic root into the coronary arteries) and/or a retrograde route (via the coronary sinus, great heart vein).

Suitable forms for oral administration include, but are not limited to, tablets, powders, compressed or coated pills, dragees, sachets, hard or gelatin capsules, sub-lingual tablets, syrups, and suspensions. Suitable forms of parenteral administration include, but are not limited to, an aqueous or non-aqueous solution or emulsion. Suitable forms for rectal administration, include, but are not limited to, suppositories with hydrophilic or hydrophobic vehicles. For topical administration, suitable forms include, but are not limited to, suitable transdermal delivery systems known in the art, such as patches, and for nasal delivery, suitable forms include, but are not limited to, aerosol and nebulized delivery systems known in the art.

A composition of the present invention (e.g., a pharmaceutical composition) may contain one or more excipients or adjuvants. Selection of excipients and/or adjuvants and the amounts to use may be readily determined by the formulation scientist upon experience and consideration of standard procedures and reference works in the field.

By “parenteral” is meant intravenous, subcutaneous or intramuscular administration. In the methods of the present invention, the composition or compound may be administered alone, simultaneously with one or more other compounds, or the composition and/or compounds may be administered sequentially, in either order. It will be appreciated that the actual method and order of administration will vary according to, inter alia, the particular preparation of compound(s) being utilized, the particular formulation(s) of the one or more other compounds being utilized, and the conditions to be treated. The optimal method and order of administration of the compounds of the disclosure for a given set of conditions can be ascertained by those skilled in the art using conventional techniques and in view of the information set out herein.

In prophylactic applications, pharmaceutical compositions or medicaments are administered to a subject susceptible to, or otherwise at risk of, occlusion or narrowing of an artery and/or its branches and/or a disease, disturbance and/or pathological condition of an artery and/or its branches in an amount sufficient to eliminate or reduce the risk, lessen the severity, or delay the onset, including biochemical, histologic and/or physiologic symptoms. In therapeutic applications, compositions or medicants are administered to a subject suspected of, or already having, occlusion or narrowing of an artery and/or its branches and/or has had or is having a disease, disturbance and/or pathological condition of an artery and/or its branches in an amount sufficient to treat, or at least partially reduce or arrest, the symptoms (biochemical, histologic and/or physiological). An amount adequate to accomplish therapeutic or prophylactic treatment is defined as an effective amount or a therapeutically or prophylactically effective dose. In either prophylactic or therapeutic regimens, compounds and/or compositions of the present invention can be administered in several doses until a desired effect has been achieved.

An effective dose or effective doses of the compositions of the present invention, for the treatment of the conditions described herein can vary depending upon many different factors, including means of administration, target site, physiological state of the subject, whether the subject is human or an animal, other medications administered, and/or whether treatment is prophylactic or therapeutic. In some embodiments, the subject is a human but nonhuman mammals including transgenic mammals can also be treated. Treatment dosages can be titrated to optimize safety and efficacy. Generally, an effective amount of the compositions of this invention will be determined by the age, weight and condition or severity of disease or disorder of the subject.

Generally, dosing (e.g., an administration) can be one or more times daily, or less frequently, such as once a day, once a week, once a month, once a year, to once in a decade, etc. and may be in conjunction with other compositions as described herein.

The dosage and frequency of administration can vary depending on whether the treatment is prophylactic or therapeutic. In prophylactic applications, a relatively low dosage can be administered at relatively infrequent intervals over a long period of time. In therapeutic applications, a relatively high dosage at relatively short intervals is sometimes appropriate until severity of the injury is reduced or terminated, and typically until the subject shows partial or complete amelioration of symptoms of injury. Thereafter, the subject can be administered a prophylactic regimen.

The terms “increased risk” and “decreased risk” as used herein define the level of risk that a subject has of an occlusion or narrowing of an artery and/or its branches and/or a disease, disturbance and/or pathological condition of an artery and/or its branches, as compared to a control subject.

A sample of this invention can be the photomicrograph of a subject's outer retinal circulation obtained with a retinal ophthalmoscope and camera device that has been subsequently digitally binarized, segmented, and selected retinal patterning metrics derived as described in Prabhakar et al. (“Genetic variation in retinal vascular patterning predicts variation in pial collateral extent and infarct volume after middle cerebral artery occlusion” Angiogenesis 18:97-114 (2014)) and as would be well known to one of ordinary skill in the art. Non-limiting examples of a sample of this invention include a listing for the above subject of values for retinal area, vessel diameter D0, vessel diameter D2, optimality, branch angle, central retinal artery equivalent (CRAE), average length of branch segments, kurtosis of distribution of branch segment lengths, and lacunarity.

As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

The present invention is more particularly described in the following examples that are intended as illustrative only since numerous modifications and variations therein will be apparent to those skilled in the art.

### EXAMPLES

The retinal circulation stands alone as a candidate tissue. It is arranged in two-dimensions which aids geometric analysis and can be imaged directly and non-invasively in humans. Moreover, sophisticated methods have been developed for quantifying the geometry of its branch-patterning. In addition, it is well known that the formation of the retinal and of neocortical vasculatures share many anatomic similarities during development and maturation, as well as structural and topographic changes that occur with aging, cardio/cerebrovascular diseases, and hereditary angiopathies. Unfortunately, since the inner retinal circulation in human and mouse lacks collaterals, it is not possible to simply quantify collaterals in retina to determine if their number and diameter predict collateral extent in brain and other tissues.

The purpose of this study was to determine if branch-patterning of retinal arterial trees varies with genetic background and correlates with differences in pial collateral extent, and in turn, infarct volume following major coronary artery (MCA) occlusion. We tested this hypothesis in a genetically diverse cohort of mice previously shown to have wide differences in collateral extent. A comprehensive set of retinal patterning metrics was examined, together with identification of several novel and more predictive metrics that define genetic-dependent retinal vascular complexity. Multivariate stepwise regression modeling was used to analyze the data in an unbiased manner. K-fold regression analysis was then used to reduce statistical bias and simulate external validation, thus improving the validity of the results. We also examined the same question for branch-patterning of the MCA tree, to determine whether the findings in retina extend to another tissue and thus offer insights into the underlying mechanism for the association.

### Pial Collateral Number and Diameter, Retinal Vascular Imaging, Infarct Volume, MCA Imaging

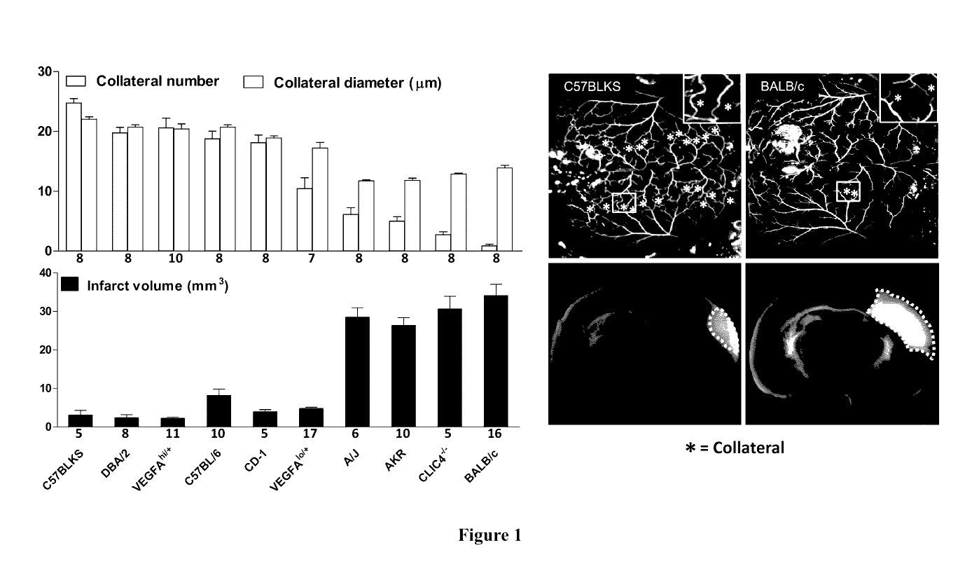

The number and average diameter of pial collaterals (COL-N, COL-D) that cross-connect the MCA and anterior cerebral artery (ACA) trees of both hemispheres were obtained from a population of ˜3 month-old male mice (n=81, ˜8 per strain) composed of 10 strains that differ widely in collateral extent (FIG. 1)^{3,4,16,18-20} We have previously found no sexual dimorphism for genetic-dependent differences in pial collateral number and diameter in different strains of classic inbred, F1 or F2 crossed, or congenic mice. The deficient strains were: VEGFA^{lo/+} A/J, AKR/J, CLIC4^{-/-}, and BALB/cByJ; the abundant strains were: C57BLKS/J, DBA/2J, VEGFA^{hi/+}, C57Bl/6J, and the CD1/CR strain (background strain for VEGFA^{lo/+}, VEGFA^{hi/+}, and CLIC4^{-/-}).

The strains VEGFA^{lo/+}, CLIC4^{-/-}, VEGF^{hi/+} and CD1 are closely related, although the gene targeted strains were generated from separate lines of CD1 which itself is maintained outbred. We selected them as part of the 10-strain population because we have shown previously that VEGF-A and CLIC4 are important in collaterogenesis, which occurs late in gestation and early postnatally, and are thus determinants of collateral number and diameter in adult and infarct volume after middle cerebral artery ligation and severity of hindlimb ischemia after femoral artery ligation; these proteins also regulate formation and patterning vessels of the general arterio-venous circulation. That is, these strains provided additional strains, besides the six classic inbred strains, with low, intermediate and high collateral number and diameter in their tissues. Although C57BL/6J (B6) and C57BLKS/J are also closely related genealogically, approximately 71% of the latter's genome derives from B6, 25% from DBA/2J and 4% from C57BL/10J, a 129 source and an unidentified source(s) (jaxmice.jax.org/strain/000662). Before the above procedures, one retina was collected from each mouse, flat-mounted, and stained (Alexafluor 568 GS-IB4). Other mice of the above strains received permanent occlusion of the right MCA trunk (MCAO), followed by determination of infarct volume 24 h later using 2,3,5-triphenyltetrazolium chloride staining. For the MCA study, the MCA artery tree was imaged in 5-6 mice of a subset of the above strains (AKR/J, BALB/cByJ, C57BLKS/J and C57Bl/6J). Different mice were used for the retina and MCA studies.

### Vascular Patterning Metrics

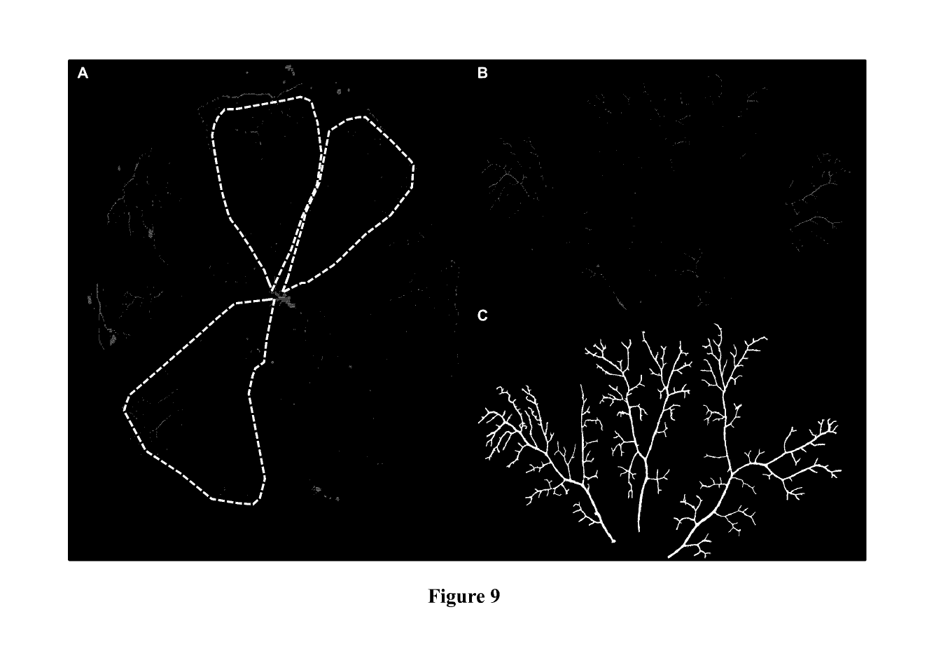

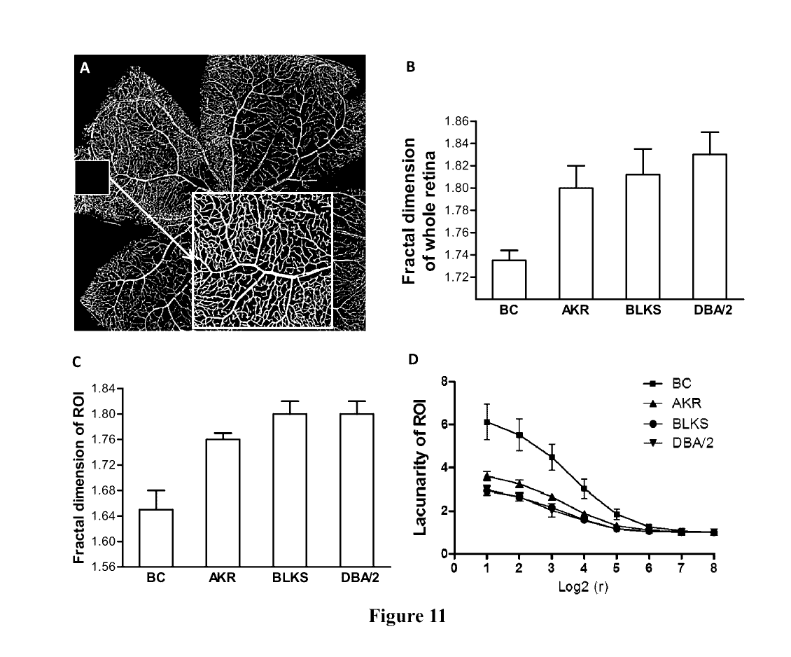

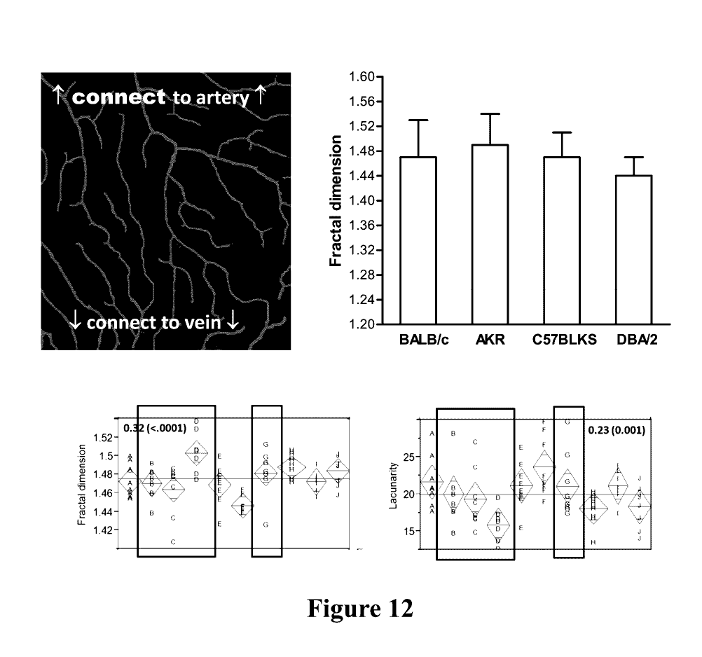

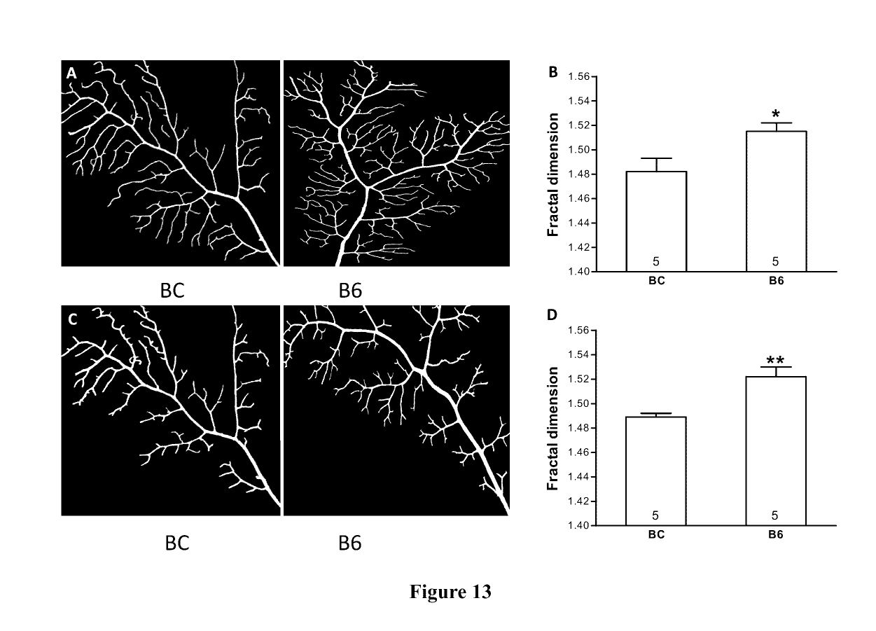

We obtained 22 retinal patterning metrics (RPMs) to define complexity of the retinal artery trees. The metrics were determined in an inner zone region extending from the optic disc margin, and an outer zone region extending from there to the retinal periphery (FIGS. 2-3, FIG. 9). The RPMs included several metrics, e.g., vessel caliber, branch angle, central retinal artery and vein equivalent (CRAE and CRVE), artery-to-vein ratio (AVR), optimality, arterial tree area, fractal dimension and lacunarity (FIG. 1, Table 1, FIG. 9). We also measured additional RPMs that identify distribution (average, skewness, kurtosis) of length and tortuosity of branch segments. Measurements were limited to arterial trees because a preliminary analysis showed that the capillary bed, venous trees, and the distal-most arteriole branches beyond the 4^{th}-order level lacked detectable strain-dependent differences in patterning and thus increased the noise in the data (FIGS. 11-13). A subset of the above metrics that were found to be most predictive in the retina study was obtained in the MCA study using the same methods for deriving the metrics as described herein. All mice were coded to assure investigators remained blinded to mouse strain and collateral extent until all measurements had been obtained.

### Image Analysis

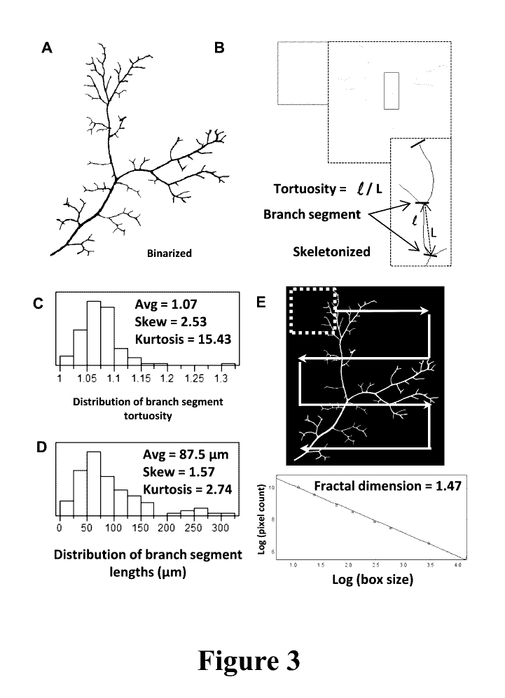

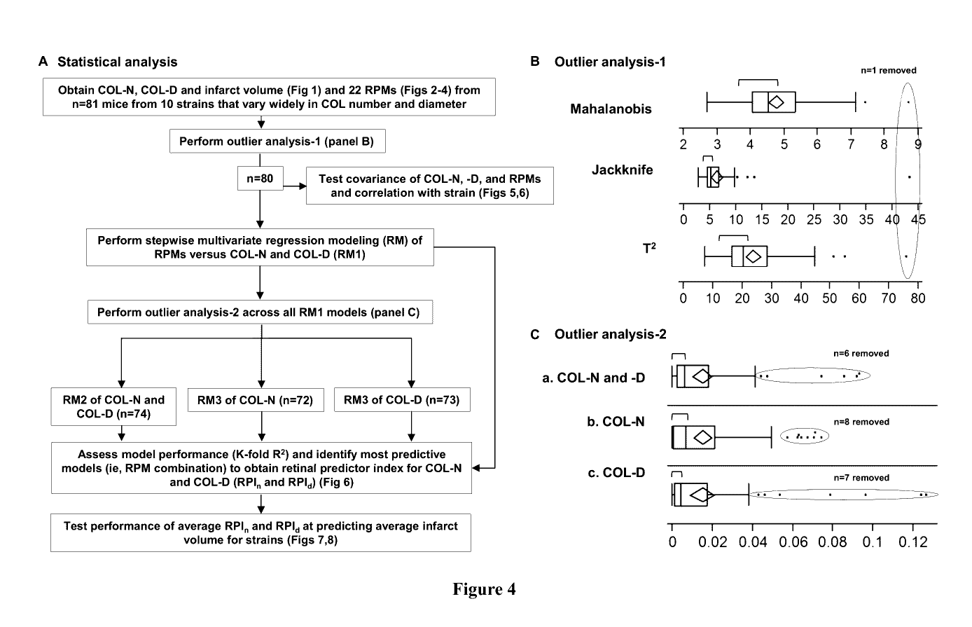

Three arterial trees were randomly chosen for each retina among the 5-7 trees present (Research Randomizer, randomizer.org). Ten RPMs were manually obtained from the inner-zone between the optic disc margin and inner zone margin, the latter defined as lying at 1/10th and 5/10th of the outer-zone margin (FIG. 2). The remaining 12 RPMs (FIG. 3) were obtained after binarization of the arterial trees (FIG. 11), followed by semi-automatic application of Image J functions, a skeletonize plugin, and a MATLAB algorithm for measuring fractal dimension and lacunarity. A highly customizable macro recorder (jitbit.com/macro-recorder) was used to create a variety of repetitive sequences of ImageJ commands and plugins followed by data-transfers to Microsoft Excel. The MATLAB code used to calculate fractal dimension and lacunarity is in Appendix 1. These action sequences were looped to allow more efficient acquisition of data from batches of a large number of images in a semi-automatic fashion.

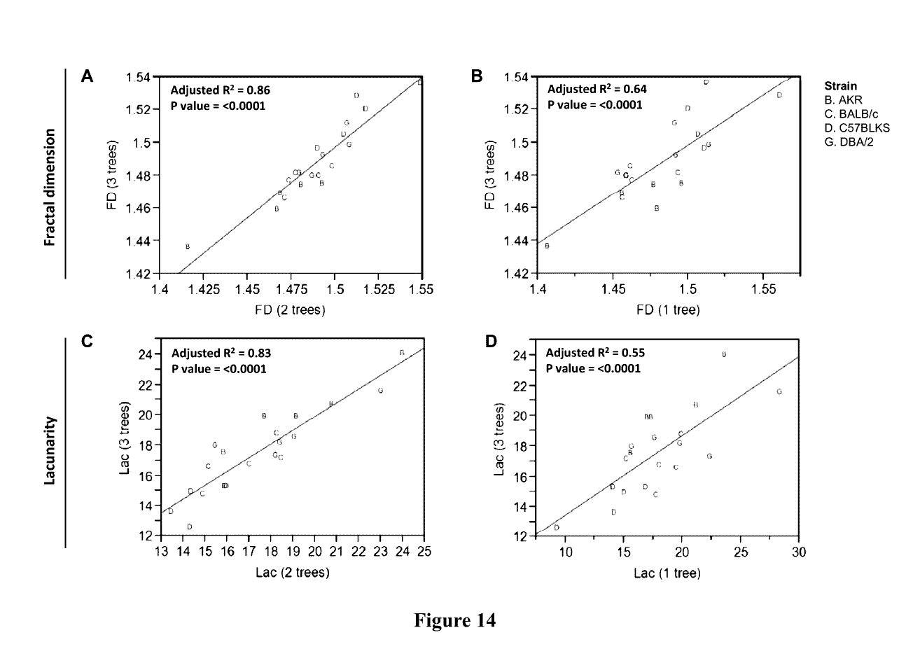

Preliminary results from 20 mice (5 each from 4 strains) indicated that analyzing 2 randomly chosen trees provided an optimal tradeoff between accuracy and time required for image binarization (FIG. 14). Therefore, images were background-corrected using Photoshop CS5, and for the remaining 61 mice, 2 instead of 3 arterial trees from each retina were randomly chosen, binarized, and capillaries and branches beyond the 4th order vessel level were removed (FIG. 11). RPMs across all measured arterial trees for a given retina were averaged.

### Study Design and Statistical Analysis

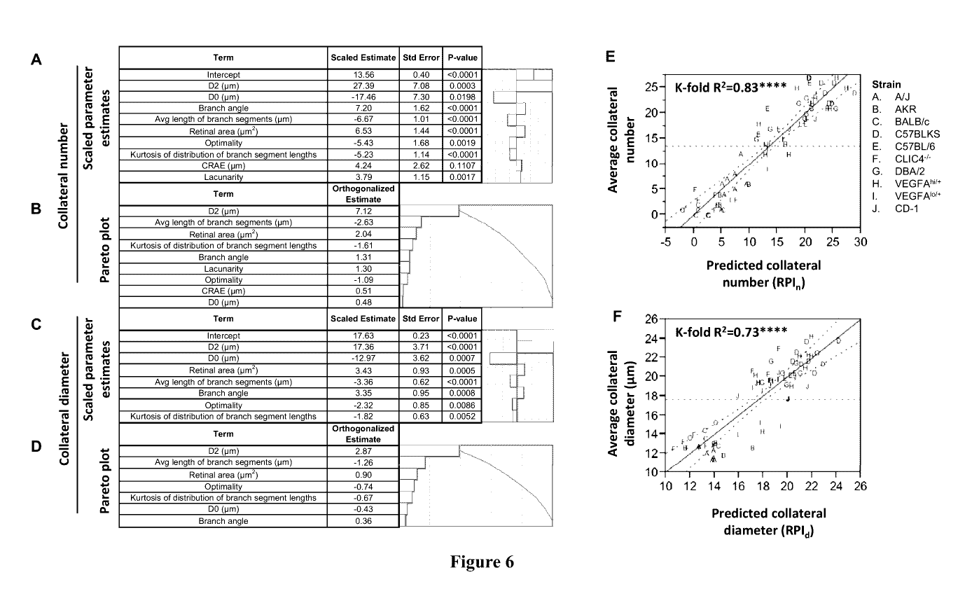

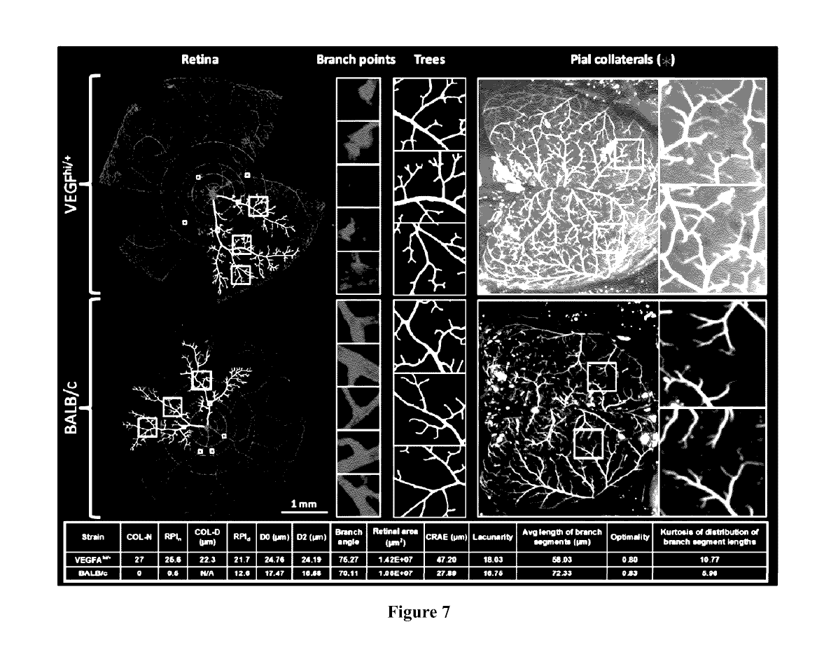

Statistical analyses were performed in JMP 9.0 (SAS, Cary, N.C.) and Microsoft Excel 2010 (FIG. 4A). After obtaining COL-N, COL-D and the 22 RPMs for each mouse, an outlier analysis was conducted using Mahalanobis, T-squared and jackknife distances, resulting in identification of one common outlier (FIG. 4B). ANOVA was then used to determine if RPMs vary with COL-N, COL-D and genetic background (FIG. 5). Finally, to test the hypothesis that RPMs predict COL-N and COL-D, stepwise multivariate regression modeling was performed on the 80 remaining mice using 3 different stopping rules [minimum Akaike's Information Criterion (AIC), minimum Bayesian Information Criterion (BIC), and p-value cut-offs] in forward, backward, and mixed directions to obtain a set of 7 regression models for both COL-N and COL-D (RM1) (FIG. 15). As an additional outlier analysis, mice with a significantly high Cook's D influence across RM1 of both COL-N and COL-D were identified and excluded (FIG. 4C). The aforementioned modeling process was then repeated to obtain 2 additional sets of regression models (RM2 and RM3) (FIG. 15). Leave-one-out K-fold cross-validation was performed³² for all 21 models for COL-N and COL-D to test predictive performance (FIG. 15). In addition, direction and significance of independent correlation of predictive RPMs were compared across all models (FIG. 15). The most predictive models for COL-N and COL-D were further examined to: 1) determine comparative predictive ability of individual RPMs as assessed by magnitude of scaled parameters and orthogonalized estimates (FIG. 6) and 2) to obtain a formula—the Retinal Predictor Index—for collateral number and diameter (RPIₙ and RPI_{d}). To aid presentation of the relationships that were identified, retinal and pial images of two mice with a wide difference in COL-N and COL-D were visually compared with values of the most predictive metrics (FIG. 7). To test whether RPMs predict severity of stroke following MCAO, performance of RPIₙ and RPI_{d} for the different mouse strains was evaluated for prediction of infarct volume (FIG. 8).

Patterning Metrics of Retinal Arterial Trees Correlate Strongly with Genetic Background-Dependent Differences in Collateral Number and Diameter

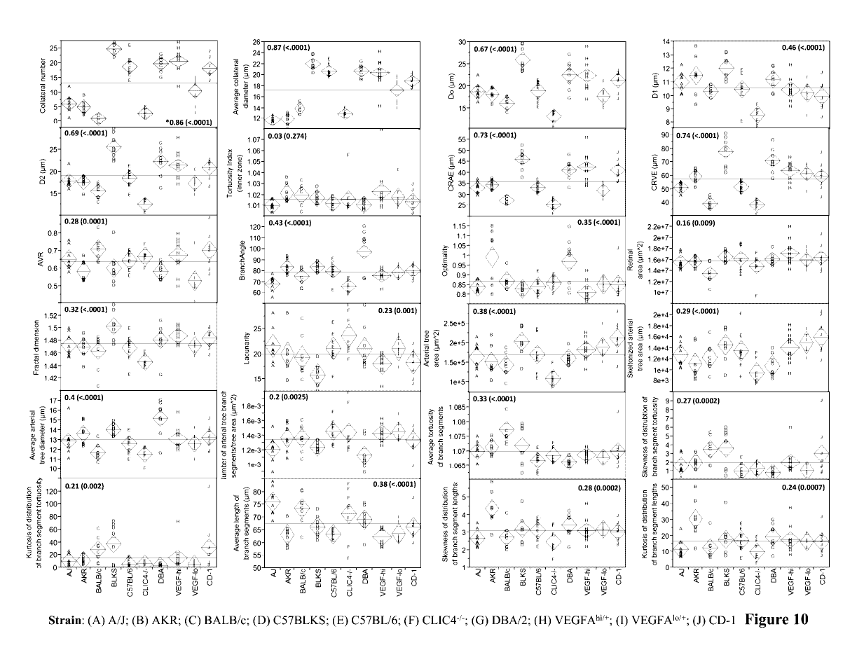

We performed a bivariate regression (ANOVA) of COL-N, COL-D, and RPMs across the 10 mouse strains (FIG. 5A, Table 1, FIG. 10). Differences in COL-N and COL-D were strongly associated with genetic background (adjusted R² of 0.86 and 0.87). Ten of the 22 RPMs varied strongly with genetic background (adjusted R²>0.35 and p<0.0001) (FIG. 5A), suggesting that, similar to the variation in COL-N and COL-D (FIG. 1), differences in genetic background cause significant differences in retinal arterial tree patterning. This is confirmed by multivariate correlation matrices (FIG. 5B,C), which show that many RPMs have a strong and significant correlation with COL-N and COL-D. These data indicate that RPMs predict collateral extent in the brain. However, the correlation matrices show that certain RPMs are also correlated with other RPMs. Thus, a step-wise, multivariate regression modeling approach was required to adjust for covariates, obtain a refined set of the most predictive RPMs, and eliminate any redundant metrics.

### Genetic-Dependent Differences in Patterning of Retinal Arterial Trees Predict Differences in Pial Collateral Number and Diameter

To determine which RPMs and in what combination most strongly predict COL-N and COL-D, we performed stepwise multivariate regression modeling (FIG. 15). The predictive performance of the models, evaluated using K-fold R² cross-validation, was strong across all models (0.73-0.83 for COL-N and 0.59-0.73 for COL-D). This indicates that genetic background plays a significant role in specifying retinal patterning and that RPMs can be used to predict genetic differences in COL-N and COL-D. Removal of outliers did not change this conclusion: K-fold R² for COL-N and COL-D only improved by an average of ˜0.05 and 0.02 for RM2 and ˜0.09 and 0.1 for RM3 (FIG. 15). In addition, all 7 models showed a similarly strong predictive performance and a nearly identical set of RPMs that were consistently predictive in the same direction, i.e., 9 RPMs for COL-N, of which 7 were also predictive for COL-D. This verifies that the findings were not dependent on or biased by a particular modeling approach. Lacunarity and CRAE were the two RPMs that predicted COL-N but not COL-D. Lacunarity only became a significant predictor across all models after removal of outliers (RM2 and RM3), and CRAE was only predictive in 4 models. These results are consistent with our finding that lacunarity and CRAE only minimally contribute to predictive performance, in comparison to the other predictors (FIG. 6). Only one of two RPMs for distribution of a metric—skewness and kurtosis of distribution of branch segment lengths—reached predictive significance across all models (i.e., kurtosis) (FIG. 15), a finding that is attributable to the strong covariance and redundancy of the two RPMs (FIG. 5B).

### A Combination of Nine Retinal Patterning Metrics Predicts Differences in Collateral Extent

To compare the relative predictive strength of the RPMs, we obtained scaled estimates and orthogonalized estimates (i.e., estimates that are set to a common relative scale and independent of other metrics after elimination of covariance) of the most strongly correlated RPMs arising from the most predictive models (identified in FIG. 15) for COL-N and COL-D (FIG. 6). The scaled estimates (FIG. 6A,C) show relative amount of change in COL-N or COL-D (in raw values) that is expected when a specific RPM changes from its lowest to highest value in the data set. However, scaled estimates only provide limited information because of covariance between certain RPMs (FIG. 5B) and because the metrics have unequal ranges and variances in the mouse cohort [e.g., optimality and fractal dimension (FD) show less variation compared to vessel caliber (FIG. 10)]. Pareto plots (FIG. 6B,D) improve the assessment of relative predictive power because they also standardize and orthogonalize the RPMs so that they have equal variances and are uncorrelated. Accordingly, the average diameter of the larger branch vessel after a branch point (D2) is the strongest predictor of collateral extent, followed closely by average length of branch segments, and retinal area. Other predictive metrics in the model, e.g., parent vessel caliber (D0), optimality ratio (a measure inversely related to equitability of distribution of blood flow from a trunk to daughter vessels at bifurcations), kurtosis of distribution (i.e., ‘peakedness’ of distribution, or “self-similarity”) of branch segment lengths, branch angle at bifurcations, retinal area, lacunarity and CRAE—although not strong contributors individually—together capture additional unique information that approximately doubles the net predictive ability beyond the three most predictive metrics. Thus, the combination of these nine features of retinal patterning strongly predicts genetic variation in collateral number and diameter. These RPMs were then combined with the most predictive models to obtain a formula—the Retinal Predictor Index—for collateral number and diameter (RPIₙ and RPI_{d}) that were then found to be strong predictors of the actual collateral number (±3.4 collaterals, K-fold R²=0.83, p<0.0001) and diameter (±1.9 μm, 0.73, p<0.0001) in each mouse (FIG. 6E,F).

FIG. 7 combines retinal and pial images with RPM data from two mice that varied widely in collateral number (27 versus 0), to address the degree to which visual versus quantitative differences in retinal patterning correlate with differences in collateral number. Consistent with the models' predictive power, a mouse with no collaterals had a predicted COL-N of 0.5, lower values for vessel calibers (D0, D2), branch angles at branch points, retinal area and CRAE, but higher lacunarity, longer branch segments, and higher optimality. However, this mouse had a lower kurtosis of distribution of branch segment lengths (i.e., lower proportion of branch segments with similar lengths)—a finding that is inconsistent with prediction and attributable to biological variance and experimental error. Also consistent with quantitative differences, the mouse with no collaterals had more acute branch angles, a smaller retinal area, lower vessel caliber overall, less vessel complexity, and fewer branch segments. On the other hand, visual differences in CRAE, optimality and kurtosis of distribution of branch segment lengths were less evident. Thus, the comparisons in FIG. 7, showing that visual differences in retinal vascular patterning are subtle in mice with even wide differences in COL-N and COL-D, underscore the need for precise quantitative measurements and multivariate regression modeling in order to predict differences in collateral extent.

### Retinal Artery Tree Geometry Predicts Infarct Volume

Differences in collateral extent are closely associated with (FIG. 1)—and causal for—differences in severity of stroke following middle cerebral artery occlusion. Given that genetic variation in retinal geometry strongly predicts variation in collateral extent (FIG. 6), we hypothesized that RPMs will also predict infarct volume (FIG. 8), using the RPIₙ and RPI_{d} derived from the above analysis. This test for association with infarct volume was necessarily conducted on a strain-wise basis, because infarct volumes for the ten strains (FIG. 1) were a combination of historic data plus addition of more animals in VEGF^{hi/+}, VEGF^{lo/+} and BALB/c strains. Average RPIs for the ten mouse strains strongly predicted average COL-N and COL-D (K-fold R² of 0.98 and 0.88, FIG. 8A). Average COL-N and COL-D among the strains strongly predicted infarct volume among the same strains (K-fold R² of 0.78 and 0.75, FIG. 8B); these values are similar to those obtained with linear correlation of collateral number and diameter against infarct volume across fifteen mouse strains (R²=0.80 and 0.71, respectively). Average values for RPIₙ and RPI_{d} among the strains thus strongly predicted average infarct volumes (K-fold R² of 0.85 and 0.87, FIG. 8C).

The flow scheme at the top of FIG. 8 derives from three observations: The data in panel A show that the average RPI for collateral number and diameter correlate closely with, i.e., “predicts (A),” the measured collateral number and diameter among the 10 strains. The data in panel B show that average collateral number and diameter also correlate well with, i.e., “predicts (B),” the measured infarct volume. The present study confirms that variation in pial collateral number and diameter are major determinants of variation in the severity of infarct volume. These data and conclusion agree with the observation that infarct volume in patients with acute ischemic stroke is strongly correlated with collateral status/score—an index of collateral-dependent flow whose major determinants are number and diameter of the pial collateral network serving the occluded territory. The data in panel C validate the inference that the RPI for collateral number and diameter therefore predict well (r²=0.85 and 0.87) the actual measured infarct volume.

Relationship of Fractal Dimension and Lacunarity with Euclidean Metrics

Fractal dimension and lacunarity are global, non-Euclidean dimensionless metrics that have been used to define complexity of the retinal vasculature in association studies. However, how they relate to geometric measures of retinal arterial tree patterning has not been determined. Since we found that differences in fractal dimension and lacunarity were associated with differences in the other patterning metrics (FIG. 5B,C; boxed-in metrics), our data set offered a unique opportunity to identify, in a complex vascular branching network, the relationship between these global metrics and the Euclidean metrics which are more intuitive and visualizable. No such analysis is extant in the literature. FIGS. 16A-I shows the association between RPMs and fractal dimension and lacunarity. As with our prior analysis, we performed forward and backward stepwise multivariate regression modeling of fractal dimension and lacunarity versus RPMs using a variety of criteria as detailed in FIG. 15. Average fractal dimension and lacunarity of retinal artery trees (dependent variables) from the 80 mice that were used above were modeled with other RPMs (independent variables). Significance of RPMs for a given model and their strength of association with fractal dimension and lacunarity, as measured by K-fold R², were identified as detailed in FIG. 6 and FIG. 15. Among RPMs that characterize retinal arterial tree complexity, high fractal dimension and low lacunarity were most strongly associated with differences in large vessel caliber (CRAE) and shorter and more tortuous branch segments, respectively.

Fractal dimension did not emerge as a significant predictor of COL-N and COL-D across most models (FIG. 15), and lacunarity only moderately predicted COL-N(FIG. 6B). The reasons for this become evident from the inspection of orthogonalized (uncorrelated, equal variance) metrics (FIGS. 16A-I), where it can be seen that these measures encompass contributions from multiple other patterning metrics that correlated either positively or negatively with collateral number and diameter (FIG. 15). Additional likely contributions to their low predictive power are the narrow range of fractal dimension (1.41-1.54) and high covariance of it and lacunarity with other RPMs (FIG. 5B, boxed-in metrics), many of which defined more specific independent features of arterial tree complexity and thus emerged as stronger predictors of collateral number and diameter.

### Middle Cerebral Artery Tree Analysis

Patterning of the MCA tree and arterial trees in skeletal muscle of C57Bl/6 mice differ qualitatively from BALB/c mice—strains with large differences in collateral number and diameter in these and other tissues. Given the above findings in the retina, we wanted to determine if patterning in another tissue would evidence similar differences and correlate with/predict differences in pial collateral extent. We studied the MCA because it can be imaged in its entirety and in quasi-2-dimension without dissection of the brain, is the largest of the cerebral artery trees, and is cross-connected to the ACA and posterior cerebral artery (PCA) by the pial collaterals, whose variation in extent is the subject of this study.

Genetic-dependent differences in arborization of the MCA tree were evident among a subset of the 10 mouse strains (BALB/c, C57BLKS, AKR and C57BL/6), when examined for the same predictive metrics that were identified in retina (FIGS. 17-19). Multivariate regression analysis showed that MCA tree geometry predicted pial collateral number and diameter, albeit with less strength than retina. However, many of the metrics correlated poorly or not at all, or with reversed slopes in bivariate regression against the same metrics in retina (FIG. 17)—findings which also undoubtedly reflect, in part, the smaller number of strains and fewer animals per strain analyzed.

The primary goal of this study was to test the hypothesis that genetic polymorphisms responsible for variation in collateral extent also cause strain-specific differences in branch-patterning of the retinal artery tree that correlate with and predict differences in collateral extent. No studies are extant in the literature that provide even an entrée to answering this question, i.e., none has examined whether retinal vascular geometry varies with genetic background in mice or other laboratory species, wherein potential confounding effects of differences in environmental factors can be held constant. Our findings support this hypothesis. Retinal artery geometry varied with differences in genetic background and strongly predicted actual pial collateral number and diameter. Values for correlation and predictive strength derived from analysis of individual mice versus across strains were (an R² of 1.00 equals a perfect correlation/prediction): R² of 0.83 versus 0.98 for number, and 0.73 versus 0.88 for diameter, with predictive strength of ±3.4 versus1.2 for number, and ±1.9 versus 1.2 μm for diameter; p<0.0001 for all (FIG. 6E,F and 8A,B). Moreover, these predicted values for collateral number and diameter strongly predicted actual infarct volume after MCA occlusion: R²=0.85 and 0.87, ±5.1 mm³ for infarct volume, p<0.0001 (FIG. 8C). This is in agreement with the major role of differences in collateral blood flow in determining differences in stroke severity. These results suggest that variation in arterial arborization in the retina may predict variation in collateral extent in humans and severity of ischemic injury if arterial obstruction occurs.

The two strongest metrics for predicting genetic differences in collateral number and diameter were D2 (diameter of the larger daughter vessel at bifurcations along the largest artery within the tree; positively correlated) and average length of branch segments (negatively correlated) (FIG. 6). The latter, which was also true in the MCA tree study, is consistent with a larger number of branches in a tree providing a greater number of opportunities for collaterals to form between adjacent trees—a process termed collaterogenesis that occurs during development. The positive relationship with D2 may reflect artery trees that form, enlarge their diameters and extend outward faster during gestation to reach the crowns of adjacent trees, thus facilitating collaterogenesis. Other patterning metrics associated with greater collateral extent and thus lesser stroke severity were: larger retinal areas, wider branch angles, more equitable distribution of blood flow (lower optimality), and greater vessel complexity (higher lacunarity, shorter branch segments, greater variation in branch segment lengths). All of these metrics describe a larger, more complex tree with a greater number of distal-most arterioles that would thus provide more opportunities to form collaterals during collaterogenesis.

The present findings demonstrate that retinal patterning predicts stroke outcome in mice. These studies also show that in mice retinal arterial tree patterning varies with genetic background and predicts genetic-dependent differences in pial collateral number, diameter and infarct volume with high accuracy (80-90%). These findings comparing retina and MCA trees suggest that the retina may be unique in the strength of its arterial geometry to predict variation in collaterals in brain and other tissues. Validation of a similar retinal predictor index in humans will lead to development of a non-invasive, relatively inexpensive biomarker to aid existing neuroimaging and hemodynamic methods, as well as possible future genetic tests, in predicting variation in collateral extent. Such a multimodal screening would provide a means to predict the likelihood of severe tissue injury before obstructive disease develops. This knowledge could aid adoption of life-styles and treatments aimed at avoiding or reducing risk factors for obstructive disease. When obstruction does occur, combining a retinal predictor index with collateral scoring would improve the knowledge needed to guide treatment choice and tailor the time-window for recanalization therapy. With progression of time after onset of stroke, patients with poor collateral status are less likely to benefit from revascularization and more likely to develop intracranial hemorrhage after treatment. Thus, such tailoring seeks to identify patients with poor collaterals for exclusion of treatment, while providing an extended window to those with good collateral circulation. In addition, stratifying patients according to collateral extent would likely reduce the variability seen in past clinical studies. Furthermore, in studies examining new treatments aimed at increasing collateral flow, variability in efficacy would likely be reduced by stratifying patients for poor versus good collateral extent.

### Detailed Materials and Methods

Pial Collateral Number and Diameter.

Brains were obtained from a population of ˜3 month-old male mice (n=81) composed of 10 strains that differ widely in collateral extent The deficient strains were: VEGFA^{lo/+} A/J, AKR/J, CLIC4^{-/-}, and BALB/cBy/J; the abundant strains were: C57BLKS/J, DBA/2J, VEGFA^{hi/+}, C57BL/6, and CD1/CR (this is the background strain for VEGFA^{w+}, VEGFA^{hi/+}, and CLIC4^{-/-}) (FIG. 9). Mice were anesthetized with ketamine and xylazine (100 and 10 mg/kg ip), heparinized, and the cerebral vasculature perfused via the thoracic aorta at 100 mmHg with phosphate buffered saline (PBS) containing 10⁻⁴M nitroprusside to produce maximal vasodilation and Evans blue to stain the endothelium. While this proceeded, a craniotomy was performed and the dorsal surface of the neocortex was treated topically with 4% paraformaldehyde (PFA) to fix the vasculature at maximal diameter. Under a stereomicroscope, the cerebral arterial vasculature was then filled with yellow MicroFil® with a viscosity set to minimize capillary transit to allow filling of the entire pial arterial circulation. After the microFil had set, the brain was fixed overnight in 4% PFA and imaged under a stereomicroscope to count the number of collaterals (COL-N) interconnecting the middle and anterior cerebral arterial (MCA, ACA) trees. The brain was then immersed in Evans blue in PBS to stain the brain parenchyma for contrast. Digital images were collected and collateral diameter (COL-D) was obtained as the average of 3 points along the center-most length of each collateral using ImageJ software. The brain was oriented to present the MCA tree in focus, and images were obtained for digital segmentation as describe below for the retina.

Mouse Retina Preparation.

Before the above procedures, retinas were collected from one eye of each of the above mice (n=81). Enucleation. Mice were anesthetized with ketamine and xylazine, and eyelids were reflected with a curved forceps. Using a stereomicroscope, the optic-nerve was severed with irridectomy scissors and the eyeball was removed and immersed in 2% PFA for 2 hours or 4% PFA for 1 hour. Eyeballs were stored at 4° C. in PBS if necessary for 8 days. Removal of retina. The eyeball was held in place with corneal side up in a Silastic-bottom glass petri dish using micropins (#26002-20, FST) passed through connective tissue attached to the sclera, and kept moist with PBS throughout the procedure. Using a 27 gauge needle, a 1 mm slit was cut at an oblique angle through the cornea at a point above the equator of the eye ball. The cornea was circumcised from the sclera by placing one blade of a Vannas scissors into the slit and hinging it on the petri dish edge, positioning the scissors tangential to the cornea and parallel to the surgical table surface, gradually cutting along the cornea's circumference while rotating the dish one whole turn in order to hemisect the eye just above the ora serrata. The lens and vitreous were gently suctioned using a micropipette followed by rinsing the retinal cup with PBS without disrupting the retina. This process was repeated 2-3 times to ensure maximum removal of the vitreous from the retinal surface. The sclera was further fastened to the silastic bottom using additional pins. The retina was gradually separated from the sclera with the ora serrata intact, using fine forceps. The retinal cups were transferred to 96 well-plates containing PBS. Staining. Following removal of PBS, retinal cups were incubated in ice cold 70% methanol for 10 minutes, rinsed with PBS 3 times for 5 minutes and incubated in PBS with 1% Triton X-100 for 30 minutes. After an additional rinse with PBS, retinal cups were incubated overnight in Alexafluor 568 GS-IB₄ (121412, Invitrogen) at 10 μg/mL in PBS on a rotator at 4° C. in the dark. Retinas were then rinsed with PBS, incubated in 1% Triton X-100 in PBS for 20 minutes, and then re-rinsed 3 times for 5 minutes in PBS.

Mouse Retina Preparation.

Mounting. Retinal cups were carefully lifted with fine forceps and placed onto Superfrost-Plus charged slides in a PBS bubble within a Pap-Pen-marked hydrophobic boundary. Using the Vannas scissors, four deep cuts were made along the circumference of the cup, extending from the ora serrata towards the optic nerve opening in order to sufficiently flatten the retina. Flattened retina was mounted onto another Superfrost-Plus slide with Vectashield under a coverslip, which was sealed with fingernail polish. Imaging. Slides were stored in paper folders in the dark at 4° C. and imaged and optically flattened using a 10× objective lens on a Nikon Surveyor microscope within 5 days of cover-slipping.

Infarct Volume.

Permanent occlusion of the right MCA trunk by micro-cautery midway between the zygomatic arch and the pinna of the ear was done on different mice from those used for the above procedures. Briefly, mice were anesthetized with ketamine and xylazine and maintained at 37° C. rectal temperature. A 4 mm skin incision was made, the midpoint of the temporal muscle separated, and a 2 mm burr-hole was made over the trunk of the MCA. The MCA was cauterized and transected, the incision closed, cephazolin and buprenorphine administered, and mice were maintained at 37° C. rectal temperature until awake. After an overdose of ketamine (100 mg/kg ip) and xylazine (15 mg/kg ip) 24 hours later, brains were removed and cooled on dry ice until the tissue became stiff, and 1 mm coronal slices were incubated in 1% 2,3,5-tripenyltetrazolium chloride in PBS at 37° C. for 20 minutes, then fixed with 1% PFA overnight. Infarct volume was calculated as the sum of the unstained volumes and expressed as a percent of total right cortical volume.

### Appendix 1

Fractal Analysis.

In fractal analysis, the raw images were first preprocessed with a manually set threshold intensity to generate the binary versions showing the vessels with intensity 1 and background with intensity 0. The fractal dimensions (FD) and the lacunarities (L) of the images were calculated with the box-counting and the gliding box algorithms respectively. Both the algorithms were implemented by home matlab codes (or routines) (Mathwork, Boston, Mass.). In box-counting for FD evaluation, the fraction of area of the image which has intensity of 1 is calculated over a range of spatial scales. At a given scale (s), the image is covered by tiled squares, and the number of squares containing at least one pixel with intensity 1, N(s) is enumerated. At the smallest scale, which is when the square size is 1×1 pixels, the number of boxes containing intensity 1 equals the number of non-zero pixels in the image. Then, in each subsequent enumeration the box size is increased by a factor of 2 until the size, s of the box equals half the size of the image. Therefore if the size of the image is 2^{k}, where k is an integer, then the number of scales at which N(s) is measured is k−1. In the final step, the slope of the best fit line through the graph of log N(s) vs log(1/s), which is FD, is calculated.

For the calculation of lacunarity, L(s), a specific scale, s is chosen. Then, a square of dimension s pixels is moved over the image pixel by pixel. At each position, the number of pixels inside the square with intensity 1 is enumerated. The mean (mean(s)) and variance (var(s)) of the number of pixels with intensity 1 from across all the positions was calculated and L(s) was determined using the following expression: 1+var(s)/mean²(s). A value of 2 pixels was chosen for s in this study because of the high sensitivity of L(s) to small box size.

The Matlab functions developed for this study are:

```
FDarbImg(inputImg)
LacArbImgBox2(inputImg)
f,idx,flag]=islist(s,nbox)
The Matlab source codes of the three functions are given below.
------------
function FD=FDarbImg(thr,inputImg)
%This program calculates the factal dimension of the input image in most
%formats
%input: thr, a value of range 0-255 segment the input image
%input: an image of vascular structures
%output: a value of fractal dimension of the input image
%codes were developed on 2013 version of Matlab platform
%Matlab image processing tool box is required
%function developed by De Chen and Stephen Lockett
%Optical Microscopy and Analysis Laboratory
%Leidos Biomedical Research, Inc.
%Frederick National Laboratory for Cancer Research
%Frederick, Maryland 21702
%version 2014, free use to all scientific users
%please report any bug to chend2@mail.nih.gov, or
locketts@mail.nih.gov
x=imread(inputImg);
[M,N]=size(x);
x=x(:,:,1);%the initial image used is in red RGB, any gray scale image will
work
[Ir,Jr]=find(x);
%detect object area of image
Len=length(Ir); %thr=180;
l=1;
while (l<Len)
  if x(Ir(l),Jr(l))<thr%180
    x(Ir(l),Jr(l))=0;
  end
  l=1+1;
end
x=im2bw(x);
%resize image
[Ir,Jr]=find(x);
[mx,t]=size(Ir);
Irs=sort(Ir);Jrs=sort(Jr);
iup=Irs(1); ilow=Irs(mx);
jup=Jrs(1); jlow=Jrs(mx);
H=ilow−iup+1;
W=jlow−jup+1;
y=zeros(H,W);
for i=1:H
  for j=1:W
    y(i,j)=x(iup−1+i,jup−1+j);
  end
end
clear x
x=y;
clear y
figure; imshow(x)
%calculate the Fractal dimension
m=min(H,W);
s=1;
while (s<=m)
  box=0;
  for i=1:s:H
    for j=1:s:W
      y=0;
      for l=1:s
        for q=1:s
          if (i+1−1<=H & j+q−1<=W)
            if x(i+1−1,j+q−1)~=0
              y=y+1;
            end
          end
        end
      end
      if y>0
        box=box+1;
      end
    end
  end
  Ns(log2(s)+1,1)=(log2(1/s));
  Ns(log2(s)+1,2)=log2(box);
  s=s*2;
end
%figure; plot(Ns(:,1),Ns(:,2),‘bo’);
r=corrcoef(Ns(:,1),Ns(:,2));
R=r(1,2);
p=polyfit(Ns(:,1),Ns(:,2),1);
FD=p(1);
end
--------------
function L=LacArbImgBox2(thr,inputImg)
%This function calculates the lacunarity of an input image of most formats
%at a fixed gliding box of size 2.
%input: thr, a value of range 0-255 segment the input image
%input: inputImg, an image of vascular structures
%output: a value of lacunarity of the input image
%codes were developed on 2013 version of Matlab platform
%Matlab image processing tool box is required
%function developed by De Chen and Stephen Lockett
%Optical Microscopy and Analysis Laboratory
%Leidos Biomedical Research, Inc.
%Frederick National Laboratory for Cancer Research
%Frederick, Maryland 21702
%version 2014, free use to all scientific users
%please report any bug to chend2@mail.nih.gov, or
locketts@mail.nih.gov
Im=imread(inputImg);
[M,N]=size(Im);
I=Im(:,:,1);%the initial image used is in red RGB, any gray scale image
will work
[Ir,Jr]=find(I);
%detect object area of image
Len=length(Ir);
l=1; %thr=180;
while (l<Len)
  if I(Ir(l),Jr(l))<thr
    I(Ir(l),Jr(l))=0;
  end
  l=1+1;
end
I=im2bw(I);
%resize image
[Ir,Jr]=find(I);
[mx,t]=size(Ir);
Irs=sort(Ir);Jrs=sort(Jr);
iup=Irs(1); ilow=Irs(mx);
jup=Jrs(1); jlow=Jrs(mx);
H=ilow−iup+1;
W=jlow−jup+1;
y=zeros(H,W);
for i=1:H
  for j=1:W
    y(i,j)=I(iup−1+i,jup−1+j);
  end
end
clear I
I=y;
clear y
[m,n]=size(I);
M=max(m,n);
r=2;%initial size of gliding box
box=[ ];
for i=1:(m−r+1)%position of gliding box
  for j=1:(n−r+1)
    S=0;
    for li=1:r
      for lj=1:r
        if I(i+li−1,j+lj−1)~=0
          S=S+1;
        end
      end
    end
    box(i,j)=S;
  end
end
ns=box;
%calculate moment
r=r/2;
N=(m−r+1)*(n−r+1);
%get the n(S,r) value
[s,z]=size(ns);
x=ns;
nbox=[ ];
for i=1:s
  for j=1:z
    if i<=m & j<=n
      S=x(i,j);
      [f,idx,flag]=islist(S,nbox);
      if f==0 & (flag==1 | flag==0)
        nbox(idx+1,1)=S;
        nbox(idx+1,2)=1;
      elseif f==1
        nbox(idx,2)=nbox(idx,2)+1;
      end
    end
  end
end
nr=nbox;
%Calculate probability distribution of the mass
y=nr;
Qs=y(:,2)./N;
Z1=sum(y(:,1).*Qs);
Z2=sum((y(:,1).*y(:,1)).*Qs);
L=Z2/(Z1)\hat{ }2;
end
--------------
function [f,idx,flag]=islist(s,nbox)
%function to determine if an passed in value S is in the list or not
%if is in the list, return a count number, otherwise return an index
%codes were developed on 2013 version of Matlab platform
%function developed by De Chen and Stephen Lockett
%Optical Microscopy and Analysis Laboratory
%Leidos Biomedical Research, Inc.
%Frederick National Laboratory for Cancer Research
%Frederick, Maryland 21702
%version 2014, free use to all scientific users
%please report any bug to chend2@mail.nih.gov, or
locketts@mail.nih.gov
if is empty (nbox)
  f=0;
  idx=0;
  flag=1;%nbox is empty
  return;
else
  [q,t]=size(nbox);
  flag=0;%nbox is not empty
end
for i=1:q
  if s==nbox(i,1)
    f=1;
    idx=i;
    flag=0;
    return;
  end
end
f=0;
idx=q;
flag=0;
```

1. A method of determining a retinal predictor index (RPI) for a tissue of interest of a subject, comprising:
a) obtaining an image of the vascular architecture of the subject's retinal circulation;
b) determining a value for the following patterning metrics of retinal artery trees in the image:
1) retinal area,
2) vessel diameter D0,
3) vessel diameter D2,
4) optimality,
5) branch angle,
6) central retinal artery equivalent (CRAE),
7) average length of branch segments,
8) kurtosis of distribution of branch segment lengths, and
9) lacunarity;
c) calculating, based on ones of the values of the patterning metrics, a retinal predictor index for collateral number (RPIₙ) and a retinal predictor index for average collateral diameter (RPI_{d}) for the subject; and
d) calculating a retinal predictor index (RPI) that is a function based on the RPIₙ and RPI_{d}, wherein the RPIₙ corresponds to and/or predicts the collateral number in the tissue of interest and the RPI_{d} corresponds to and/or predicts the average collateral diameter in the tissue of interest.

2. The method of claim 1, wherein the tissue of interest is selected from the group consisting of brain, spinal cord, heart, lung, abdominal organ, upper extremity, lower extremity, skin, skeletal muscle, bone and any combination thereof.

3. The method of claim 1, further comprising assessing the subject's demographics, clinical parameters and/or medical history and factoring them with the RPI to determine a course of medical and/or surgical treatment.

4. The method of claim 1, wherein calculating the retinal predictor index n (RPIₙ) comprises calculating the retinal predictor index n (RPIₙ) for the subject using the vessel diameter D2, the average length of branch segments, the retinal area, the kurtosis of distribution of branch segment lengths, the branch angle, the lacunarity, the optimality, the central retinal artery equivalent (CRAE), and the vessel diameter D0.

5. The method of claim 4, wherein the retinal predictor index n (RPIₙ) comprises a sum of:
a summative constant j;
a product of the vessel diameter D2 and a coefficient a;
a product of the average length of branch segments and a coefficient b;
a product of the retinal area and a coefficient c;
a product of the kurtosis of distribution of branch segment lengths and a coefficient d;
a product of the branch angle and a coefficient e;
a product of the lacunarity and a coefficient f;
a product of the optimality and a coefficient g;
a product of the CRAE and a coefficient h; and
a product of the vessel diameter D0 and a coefficient k,
wherein the summative constant j is in a range of about −4.0 to about 12.0,
wherein the coefficient a is in a range of about 2.0 to about 6.0,
wherein the coefficient b is in a range of about −1.0 to about 1.0,
wherein the coefficient c is in a range of about 1.0*10⁵ to about 1.0*10⁻⁸,
wherein the coefficient d is in a range of about −1.0 to about 1.0,
wherein the coefficient e is in a range of about 0.10 to about 0.40,
wherein the coefficient f is in a range of about 0.25 to about 0.70,
wherein the coefficient g is in a range of about −19.0 to about −36.0,
wherein the coefficient h is in a range of about 0.05 to about 0.50, and
wherein the coefficient k is in a range of about −3.0 to about 3.0.

6. The method of claim 5,
wherein the summative constant j is about 4.91±17.2 (standard error of 8.80);
wherein the coefficient a is about 2.91±1.47, (standard error of 0.75);
wherein the coefficient b is about −0.511±0.151, (standard error of 0.08);
wherein the coefficient c is about 1.1*10⁻⁶±4.95e-7, (standard error of 2.52*10-7);
wherein the coefficient d is about −0.268±0.114, (standard error of 0.058);
wherein the coefficient e is about 0.222±0.098, (standard error of 0.050);
wherein the coefficient f is about 0.443±0.265, (standard error of 0.135);
wherein the coefficient g is about −27.3±16.5, (standard error of 8.41);
wherein the coefficient h is about 0.262±0.318, (standard error of 0.161); and
wherein the coefficient k is about −1.96±1.61, (standard error of 0.820).

7. The method of claim 1, wherein calculating the retinal predictor index d (RPI_{d}) comprises calculating the retinal predictor index d (RPI_{d}) for the subject using the vessel diameter D2, the average length of branch segments, the retinal area, the optimality, the kurtosis of distribution of branch segment lengths, the vessel diameter D0, and the branch angle.

8. The method of claim 7, wherein the retinal predictor index d (RPI_{d}) comprises the sum of:
a summative constant m;
a product of the vessel diameter D2 and a coefficient n;
a product of the average length of branch segments and a coefficient p;
a product of the retinal area and a coefficient q;
a product of the optimality and a coefficient r;
a product of the kurtosis of distribution of branch segment lengths and a coefficient s;
a product of the vessel diameter D0 and a coefficient t; and
a product of the branch angle and a coefficient u,
wherein the summative constant m is in a range of about 10.0 to about 30.0,
wherein the coefficient n is in a range of about 0.5 to about 3.5,
wherein the coefficient p is in a range of about −0.05 to about −0.40,
wherein the coefficient q is in a range of about 5.0*10- to about 5.0*104,
wherein the coefficient r is in a range of about −1.0 to about −20.0,
wherein the coefficient s is in a range of about −0.005 to about −0.15,
wherein the coefficient t is in a range of about −2.5 to about 0.01, and
wherein the coefficient u is in a range of about 0.01 to about 0.20.

9. The method of claim 7,
wherein the summative constant is about 20.3±8.51 (standard error of 4.34),
wherein the coefficient n is about 1.79=0.751 (standard error of 0.383),
wherein the coefficient p is about −0.229±0.082 (standard error of 0.042),
wherein the coefficient q is about 5.4*10⁻⁷±2.86e-7 (standard error of 1.46e-7),
wherein the coefficient r is about −11.6±8.41 (standard error of 4.29),
wherein the coefficient s is about −0.0930±0.063 (standard error of 0.032),
wherein the coefficient t is about −1.37±0.747 (standard error of 0.381), and
wherein the coefficient u is about 0.103±0.057 (standard error of 0.029).

10. The method of claim 1, wherein calculating the RPI comprises performing a mathematical operation on RPI, and RPI_{d}.

11. The method of claim 1, further comprising determining a value for the retinal patterning metrics:
1) fractal dimension,
2) arterial tree area,
3) skeletonized arterial tree area,
4) average arterial tree diameter,
5) number of arterial tree branch segments/tree area,
6) tortuosity index (inner zone),
7) skewness of distribution of branch segment tortuosity,
8) kurtosis of distribution of branch segment tortuosity,
9) average length of branch segments,
10) skewness of distribution of branch segment lengths, and/or
11) central retinal artery-to-vein ratio (AVR).

12. The method of claim 1, further comprising determining a value for the retinal patterning metrics:
1) Branch lengths distribution points: Branch lengths maximum,
2) Branch lengths distribution points: Branch lengths minimum,
3) Branch lengths distribution points: Branch lengths 25^{th} percentile,
4) Branch lengths distribution points: Branch lengths 75^{th} percentile,
5) Branch lengths distribution points: Branch lengths median,
6) Tortuosity of branches distribution points: Tortuosity maximum,
7) Tortuosity of branches distribution points: Tortuosity minimum,
8) Tortuosity of branches distribution points: Tortuosity 25^{th} percentile,
9) Tortuosity of branches distribution points: Tortuosity 75^{th} percentile,
10) Tortuosity of branches distribution points: Tortuosity median,
11) Average tortuosity of branch segments,
12) Number of bifurcations per tree,
13) Number of trees crossing the optic disc demarcator,
14) Number of trees crossing the inner zone margin,
15) Percent area skeletonized on area canvas used to obtain fractal dimension and lacunarity (e.g., 25×25),
16) Total length based on Image J analyze skeleton plugin,
17) Average diameter,
18) Number of branches,
19) Number of junctions,
20) Number of end-points,
21) Average branch length from calculated total,
22) Average branch length from analyze skeleton plugin based total length,
23) N number of branches,
24) N number of junctions,
25) N number of end-points,
26) Hull span ratio,
27) Fractal dimension from Image J plugin, and/or
28) Lacunarity from Image J plugin.

13. The method of claim 1, where one or more of the operations are performed using at least one processor.

14. A method of identifying the likelihood of poor prognosis in a subject with occlusion or narrowing of an artery and/or its branches, comprising:
a) obtaining an image of the vascular architecture of the subject's retinal circulation;
b) determining a value for the following patterning metrics of retinal artery trees in the image:
1) retinal area,
2) vessel diameter D0,
3) vessel diameter D2,
4) optimality,
5) branch angle,
6) central retinal artery equivalent (CRAE),
7) average length of branch segments,
8) kurtosis of distribution of branch segment lengths, and
9) lacunarity;
c) calculating, based on ones of the values of the patterning metrics, a retinal predictor index for collateral number (RPIₙ) and a retinal predictor index for average collateral diameter (RPI_{d}) for the subject; and
d) calculating a retinal predictor index (RPI) that is a function based on the RPIₙ and RPI_{d}, wherein a RPI of the subject that is less than a threshold RPI identifies the subject as having an increased likelihood of poor collaterals in the tissue supplied by the occluded or narrowed artery and/or its branches and poor prognosis and a RPI of the subject that is greater than or equal to a threshold RPI identifies the subject as having an increased likelihood of good collaterals in the tissue supplied by the occluded or narrowed artery and/or its branches and good prognosis.

15. A method of producing a retinal predictor index (RPI) nomogram, comprising the steps of:
a) obtaining an image of the vascular architecture of the retinal circulation from each subject in a population of subjects;
b) determining for each image obtained from each subject in the population of (a), a value for the following patterning metrics of retinal artery trees in the image:
1) retinal area,
2) vessel diameter D0,
3) vessel diameter D2,
4) optimality,
5) branch angle,
6) central retinal artery equivalent (CRAE),
7) average length of branch segments,
8) kurtosis of distribution of branch segment lengths, and
9) lacunarity,
10) fractal dimension,
11) arterial tree area,
12) skeletonized arterial tree area,
13) average arterial tree diameter,
14) number of arterial tree branch segments/tree area,
15) tortuosity index (inner zone),
16) skewness of distribution of branch segment tortuosity,
17) kurtosis of distribution of branch segment tortuosity,
18) average length of branch segments,
19) skewness of distribution of branch segment lengths, and
20) central retinal artery-to-vein ratio (AVR);
c) identifying first key metrics of the patterning metrics of (b) for calculating a retinal predictor index n (RPIₙ) for each subject;
d) identifying second key metrics of the patterning metrics of (b) for calculating a retinal predictor index d (RPI_{d}) for each subject;
e) calculating, based on the values of the first key metrics, a retinal predictor index n (RPIₙ) for each subject;
f) calculating, based on the values of the second key metrics, a retinal predictor index d (RPI_{d}) for each subject;
g) calculating a retinal predictor index (RPI) for each subject that is a function based on the RPIₙ and RPI_{d} of each subject;
h) determining collateral blood flow for each subject; and
i) mathematically and graphically identifying the relationship between the RPI and collateral blood flow for each subject in the population in a format that establishes quintiles for the population, thereby producing the RPI nomogram.

16. A retinal predictor index (RPI) nomogram produced by the method of claim 15.

17. A method of identifying the likelihood of poor stroke prognosis in a subject in need thereof, comprising:
a) obtaining an image of the vascular architecture of the subject's retinal circulation;
b) determining a value for the following patterning metrics of retinal artery trees in the image:
1) retinal area,
2) vessel diameter D0,
3) vessel diameter D2,
4) optimality,
5) branch angle,
6) central retinal artery equivalent (CRAE),
7) average length of branch segments,
8) kurtosis of distribution of branch segment lengths, and
9) lacunarity;
c) calculating, based on ones of the values of the patterning metrics, a retinal predictor index for collateral number (RPIₙ) and a retinal predictor index for average collateral diameter (RPI_{d}) for the subject; and
d) calculating a retinal predictor index (RPI) that is a function based on the RPIₙ and RPI_{d}, wherein a RPI of the subject that is within the first or second quintile of the nomogram of claim 16 identifies the subject as having an increased likelihood of poor pial collaterals and poor stroke prognosis, and an RPI of the subject that is within the third quintile of said nomogram identifies the subject as having an increased likelihood of intermediate pial collaterals and intermediate stroke prognosis, and an RPI of the subject that is within the fourth or fifth quintile of said nomogram identifies the subject as having an increased likelihood of good pial collaterals and good stroke prognosis.

18. A computer program product, comprising:
a non-transitory computer readable storage medium storing computer readable program code that, when executed by a processor of an electronic device, causes the processor to perform operations comprising:
receiving a retinal image that corresponds to a subject and that is generated using an optical device;
extracting, binarizing and segmenting one or more of a plurality of retinal artery trees identified in the retinal image;
estimating a plurality of retinal patterning metrics corresponding to the retinal image;
calculating a retinal predictor index n (RPIₙ) that corresponds to/predicts the number of the collaterals in a tissue of interest;
calculating a retinal predictor index d (RPI_{d}) that corresponds to/predicts the average diameter of the collaterals in a tissue of interest;
calculating an retinal predictor index (RPI) score using the retinal predictor index n (RPIₙ) and the retinal predictor index d (RPI_{d}); and
comparing the RPI to a threshold RPI value.

19. The computer program product of claim 18, further comprising identifying a likelihood of poor collaterals and thus poor prognosis, or good collaterals and thus good prognosis, in a subject with stroke and/or with acute or chronic occlusion and/or narrowing of an artery and/or its branches, and/or with a disease, disturbance or pathological condition of an artery and/or its branches, responsive to comparing the RPI to a threshold RPI value.

20. A computer program product, comprising:
a non-transitory computer readable storage medium storing computer readable program code that, when executed by a processor of an electronic device, causes the processor to perform operations described in claim 1.

21. An electronic device comprising:
a user interface;
a processor; and
a memory coupled to the processor and comprising computer readable program code that when executed by the processor causes the processor to perform operations comprising:
receiving a retinal image that corresponds to a subject and that is generated using an optical device;
extracting, binarizing and segmenting one or more of a plurality of retinal artery trees identified in the retinal image;
estimating a plurality of retinal patterning metrics corresponding to the retinal image;
calculating a retinal predictor index n (RPIₙ) that corresponds to and/or predicts the collateral number in a tissue of interest;
calculating a retinal predictor index d (RPI_{d}) that corresponds to and/or predicts the average collateral diameter in a tissue of interest;
calculating a retinal predictor index (RPI) score using the retinal predictor n index (RPIₙ) and the retinal predictor index d (RPI_{d}); and
comparing the RPI to a threshold RPI value.

22. The electronic device of claim 21, further comprising an operation comprising identifying a likelihood of poor or good prognosis in a subject responsive to comparing the RPI to a threshold RPI value.
